(19) United States
(12) Patent Application Publication
(10) Pub. No.: US 2004/0242515 A1
(43) Pub. Date: Dec. 2, 2004

(54) Trp1, mct, or ftz-f1 homologous proteins involved in the regulation of energy home-ostasis

(76) Inventors: Karsten Eulenberg, Goettingen (DE); Guenter Broenner, Goettingen (DE); Thomas Haeder, Goettingen (DE); Thomas Ciossek, Goettingen (DE); Arnd Steuernagel, Goettingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/481,960

(22) PCT Filed: Jun. 26, 2002
(86) PCT No.: PCT/EP02/07079

(30) Foreign Application Priority Data
Jun. 27, 2001 (EP) 01115482.0
Jun. 29, 2001 (EP) 01115965.4
Jul. 12, 2001 (EP) 01117033.9

(51) Int. Cl. A61K 48/00; C12Q 1/68; C12N 15/85

(52) U.S. Cl. 514/44; 435/6; 435/455

(57) ABSTRACT

The present invention discloses Trp 1, MCT, or Ftz-F 1 homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of diseases and disorders related to body-weight regulation, for example, but not limited to, metabolic diseases such as obesity, as well as related disorder's such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others.

This invention relates to the use of nucleic acid and amino acid sequences of Trp1, MCT, or Ftz-F1 homologous proteins, and to the use of these sequences in the diagnosis, study, prevention, and treatment of diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others.

Obesity is one of the most prevalent metabolic disorders in the world. It is still poorly understood human disease that becomes more and more relevant for western society. Obesity is defined as an excess of body fat, frequently resulting in a significant impairment of health. Besides severe risks of illness such as diabetes, hypertension and heart disease, individuals suffering from obesity are often isolated socially. Human obesity is strongly influenced by environmental and genetic factors, whereby the environmental influence is often a hurdle for the identification of (human) obesity genes. Obesity is influenced by genetic, metabolic, biochemical, psychological, and behavioral factors. As such, it is a complex disorder that must be addressed on several fronts to achieve lasting positive clinical outcome. Obese individuals are prone to ailments including: diabetes mellitus, hypertension, coronary heart disease, hypercholesterolemia, dyslipidemia, osteoarthritis, gallstones, cancer, e.g. cancers of the reproductive organs, and sleep apnea.

Obesity is not to be considered as a single disorder but a heterogeneous group of conditions with (potential) multiple causes. Obesity is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known.

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses specific genes involved in the regulation of body-weight, energy homeostasis, metabolism, and obesity, and thus in disorders related thereto such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. The present invention describes human translocation protein 1 (Trp1), monocarboxylate transporter (MCT), and nuclear hormone receptor 1 (FTZ-F1) genes as being involved in those conditions mentioned above.

The Drosophila melanogaster gene Trp1 (Translocation protein 1) is a component of and has a conserved function in a transport protein complex (referred to as translocon) in the endoplasmic reticulum membrane. The Drosophila Trp1 gene encodes a protein that is a structural and functional homolog of the yeast endoplasmic reticulum membrane-bound translocation protein Sec62p. Expression of the Trp1 gene throughout Drosophila development is characterized by peaks in mid-embryo-genesis and mid-pupation, followed by a sustained period of mRNA accumulation in adults (Noel P. and Cartwright I. L, 1994, EMBO J. 13(22):5253-5261). The human cDNA HTP1 (for human translocation protein 1) encodes a protein of 399 amino acids that is 36.3% identical (64.6% similar) to the Drosophila homologue of Sec62p, Drosophila translocation protein 1 (Trp1). HTP1 transcripts are expressed in various human tissues such as heart, brain, placenta, liver and pancreas (Daimon M. et al., 1997, Biochem Biophys Res Commun 230(1):100-104).

Monocarboxylate transporters (MCT) are involved in the translocation of lactate, pyruvate, and other monocarboxylates and participate in the Cori cycle and a recently discovered pathway of monocarboxylate metabolism in muscle and sperm. Lactate produced by the muscle glycolysis is transported via the bloodstream to the liver, where it is converted to glucose by gluconeogenesis. The glucose is then transported back to the muscle via the bloodstream and may be stored as glycogen (Cori cycle).

MCT activity limits mitochondrial pyruvate utilization at physiological concentrations. Increased rates of the Cori cycle have been observed in obese subjects with non-insulin-dependent diabetes mellitus (NIDDM) and an effect of weight reduction has been discussed. Lactate/monocarboxylate transporter isoforms are expressed in pancreatic islets and exocrine pancreas. Diet induced ketosis increase monocarboxylate transporter (MCT1) levels in rat brain. Lactate transport in rat adipocytes is mediated by MCT1 and is modulated during streptozotocin-induced diabetes. Mutations in MCT1 cDNA have been described in patients with symptomatic deficiency in lactate transport. The low-affinity monocarboxylate transporter MCT4 is adapted to the export of lactate in highly glycolytic cells. Overexpression of monocarboxylate transporter and lactate dehydrogenase alters insulin secretory responses to pyruvate and lactate in beta cells. Cardiac and skeletal muscle mitochondria have a monocarboxylate transporter MCT1.

The Ftz-F1 (Fushi tarazu) protein is one of the evolutionarily oldest nuclear receptor types and is a conserved member of the nuclear (steroid hormone) receptor superfamily. Highly conserved homologues have been found in vertebrates (including humans) and arthropods. Conserved functions for Ftz-F1 have been implicated in the regulation of its cofactor Fushi tarazu during embryogenesis, molting and metamorphosis of Drosophila melanogaster and Caenorhabditis elegans, and Ftz-F1 is required for steroidogenesis and sexual differentiation in mice. Human Ftz-F1 shows expression in liver and digestive organs and autoregulates its expression. Human and Drosophila Ftz-F1 are essential for epidermis and gonad development and is involved in sex determination. Human Ftz-F1 activity participates in pituitary gland development and is dependent on pituitary gland control. The pituitary gland regulates food intake via hormone secretion through the activity of the HPA axis (hypothalamic-pituitary-adrenal (HPA) axis) and participates in the control of metabolism via hormones. Interestingly, changes of the activity of the HPA axis are observed in different obesity phenotypes.

The human ortholog of Drosophila FTZ-F1 protein has been described as a member of the nuclear orphan receptor family. It was proposed that FTZ-F1 is involved in the regulation of the expression of a microsomal liver protein of the cytochrome P450 family (cholesterol 7-hydroxylase, Cyp 7) which is induced by cholesterol and suppressed by bile acids (see, for example, U.S. Pat. No. 5,958,697; U.S. Pat. No. 6,027,901; U.S. Pat. No. 6,297,019). Cyp7 is the first and rate-limiting enzyme in cholesterol catabolism in liver. Cholesterol is essential for membranogenesis and synthesis of sterol and nonsterols; excess cholesterol is deposited in arteries and can leed to artherosclerosis. Mice lacking Cyp7 show abnormal lipid excretion, skin pathology and behavioral irregularities whereas homozygous mice without Cyp7 are normal at birth but die within the first 18 days. Addition of vitamins to the water of the mother prevented early death of the offspring, and addition of cholic acid prevented late death of the offspring. Cyp7−/− escapers show induction of an alternate pathway for bile acids biosynthesis (induction of oxysterol 7-hydroxylase after 21 to 30 days after birth like in wildtype). Cyp7−/− mice have no gain of body weight to normal rates, have an oily coat due to excess of monoglyceride esters, hyperkeratosis, and apparent vision defects. Additionally, Cyp7−/− mice have normal serum lipid, cholesterol, and lipoprotein contents, but low to undetectable vitamin D3 and E levels. Fat content in stool is significantly elevated in newborn Cyp7−/− mice.

So far, it has not been described that members of the gene families of translocation proteins (e.g. Trp1), monocarboxylate transporters (e.g. MCT), or nuclear hormone receptors (e.g. Ftz-F1) are involved in the regulation of energy homeostasis and thus, no functions in metabolic diseases have been discussed. In this invention we demonstrate that the correct gene doses of the Drosophila melanogaster homologues of Trp1, MCT, or Ftz-F1 are essential for maintenance of energy homeostasis in adult flies. A genetic screen was used to identify that members of the gene families of translocation proteins (e.g. Trp 1), monocarboxylate transporters (e.g. MCT), or nuclear hormone receptors (e.g. Ftz-F1) are involved in energy homeostasis in Drosophila melanogaster.

Therefore, identification of molecules related to Trp1, MCT, or Ftz-F1 as modulators of energy homeostasis satisfies a need in the art by providing new compositions useful in diagnosis, treatment, and prognosis of diseases and disorders related to energy homeostasis regulation such as metabolic diseases and dysfunctions (for example, obesity, adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia), and related disorders like osteoarthritis, gallstones, cancer, e.g. cancers of the reproductive organs, sleep apnea, and others.

Particularly, the invention relates to pharmaceutical compositions comprising a nucleic acid molecule of the translocation protein, monocarboxylate transporter, or nuclear hormone receptor gene family or a polypeptide encoded thereby or a fragment or a variant of said nucleic acid molecule or said polypeptide or an effector, e.g. an antibody, an aptamer or another receptor recognizing said nucleic acid or polypeptide together with pharmaceutically acceptable carriers, diluents and/or adjuvants.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

The present invention discloses Trp1, MCT, or Ftz-F1 homologous proteins that are regulating the energy homeostasis and the metabolism of triglycerides, and polynucleotides, which identify and encode the proteins. The invention also relates to vectors, host cells, effectors of proteins and polynucleotides, e.g. antibodies and recombinant methods for producing the polypeptides and polynucleotides of the invention. The invention also relates to the use of these sequences and effecors thereof in the diagnosis, study, prevention, and treatment of diseases and disorders related to body-weight regulation.

Trp1, MCT, or Ftz-F1 homologous proteins and nucleic acid molecules coding therefore are obtainable from insect or vertebrate species, e.g. mammals or birds. Particularly preferred are human Trp1, MCT, or Ftz-F1 homologous nucleic acids, particularly nucleic acids encoding human Trp1 protein (GenBank Accession No. NM_{—}003262 for the cDNA, NP_{—}003253 for the protein), human MCT proteins (e.g. GenBank Accession No. NM_{—}013356 for the cDNA; NP_{—}037488 for the protein, monocarboxylate transporter 3, Genbank Accession No. NM_{—}004695.2 for the cDNA, NP_{—}004686 for the protein, solute carrier family 16-monocarboxylic acid transporters-member 5), or human ftz-F1 homologous nucleic acids, particularly nucleic acids encoding a human Ftz-F1 protein (e.g. GenBank Accession No. NM_{—}003822—derived from GenBank Accession No. AF146343; GenBank Accession No. NM_{—}004959 (derived from GenBank Accession No. U76388).

Preferred examples of human ftz-F1 homologous nucleic acids and proteins coding therefor are selected from Genbank Accession No. AB019246 Ftz-F1 related protein, AF049102 α1-fetoprotein transcription factor, short variant, U93553 α1 fetoprotein transcription factor, NM_{—}003822 nuclear receptor subfamily 5, group A, member 2, U76388 steroidegenic factor 1, U80251 hepatocytic transcription factor (hB1F), AF146343 CYP7A promoter binding factor (CPF), XM_{—}001441, AF190464, AF124247, AF228413, AF112344 or fragments thereof.

Also particularly preferred are Drosophila Trp1 homologous nucleic acids and polypeptides encoded thereby (Acc. No Z38100, GadFly Acc. No. AAF52847, or GadFly Acc. No. AAF52848), Drosophila MCT-like nucleic acids and polypeptides encoded thereby (e.g. GadFly Accession Number CG8051 or Gadfly Acc. No CG3456), or Drosophila ftz-F1 homologous nucleic acids and polypeptides encoded thereby (Acc. No. M63711 for ftz-F1 alpha, Acc. No. M98397 for ftz-F1 beta).

In a preferred embodiment the present invention also comprises Zinc finger domains (Type zf-c4 in Drosophila Ftz-F1 alpha amino acids 448-523, Acc. No. AAA28542) and/or ligand binding domains (referred to as hormone_rec in Drosophila Ftz-F1 alpha amino acids 778-938) of the proteins and nucleic acid molecules coding therefor. These zinc finger domains and/or ligand binding domains may also be fused to heterologous protein domains and nucleic acids coding therefor.

The invention particularly relates to a nucleic acid molecule encoding a polypeptide contributing to the regulation of the energy homeostasis and/or the metabolism of triglycerides, wherein said nucleic acid molecule comprises

(a) the nucleotide sequence of GenBank Acc. No. Z38100, SEQ ID NO: 1 (GadFly Accession Number CG8051), GenBank Acc. No. M63711, or GenBank Acc. No. M98397 or a human homologous nucleic acid,

(b) a nucleotide sequence which hybridizes at 66° C. in a solution containing 0.2×SSC and 0.1% SDS to the complementary strand of a nucleic acid molecule encoding the amino acid sequence of SPTREMBL Acc. No. Q24559, SEQ ID NO:2, GenBank Acc. No. AAA28542, or GenBank Acc. No. AAA28915 or a human homologous nucleic protein,

(c) a sequence corresponding to the sequences of (a) or (b) within the degeneration of the genetic code,

(d) a sequence which encodes a polypeptide which is at least 85%, preferably at least 90%, more preferably at least 95%, more preferably at least 98% and up to 99.6% identical to Acc. No. Q24559, SEQ ID NO:2, GenBank Acc. No. AAA28542, or GenBank Acc. No. AAA28915 or a human homologous protein,

(e) a sequence which differs from the nucleic acid molecule of (a) to (d) by mutation and wherein said mutation causes an alteration, deletion, duplication or premature stop in the encoded polypeptide or

(f) a partial sequence of any of the nucleotide sequences of (a) to (e) having a length of at least 15 bases, preferably at least 20 bases, more preferably at least 25 bases and most preferably at least 50 bases.

The invention is based on the finding that Translocation protein 1 (herein referred to as Trp 1), monocarboxylate transporter-like (herein referred to as MCT), or Ftz transcription factor 1 (herein referred to as Ftz-F1) homologous proteins and the polynucleotides encoding these, are involved in the regulation of triglyceride storage and therefore energy homeostasis.

To find genes with novel functions in energy homeostasis, metabolism, and obesity, a functional genetic screen was performed with the model organism Drosophila melanogaster (Meigen). One resource for screening was a proprietary Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells. In order to isolate genes with a function in energy homeostasis, several thousand EP-lines were tested for their triglyceride content after a prolonged feeding period. Lines with significantly changed triglyceride content were selected as positive candidates for further analysis.

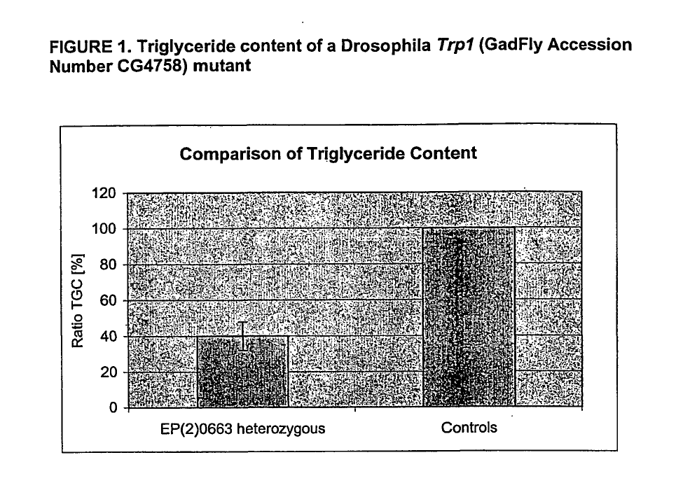

Obese people mainly show a significant increase in the content of triglycerides. In this invention, the content of triglycerides of a pool of flies with the same genotype after feeding for six days was analyzed using a triglyceride assay, as, for example, but not for limiting the scope of the invention, is described below in the EXAMPLES section. Male flies heterozygous for the integration of a vector for Drosophila line EP(2)0663, and male flies homozygous for the integration of vectors for Drosophila lines EP(X)11089, EP(3)0447, or EP(3)25823, were analyzed in an assay measuring the triglyceride contents of these flies, illustrated in more detail in the EXAMPLES section. The results of the triglyceride content analysis are shown in FIGS. 1, 4, and 9, respectively.

Genomic DNA sequences were isolated that are localized to the EP vector (herein EP(2)0663, EP(X)11089, EP(3)0447, or EP(3)25823) integration.

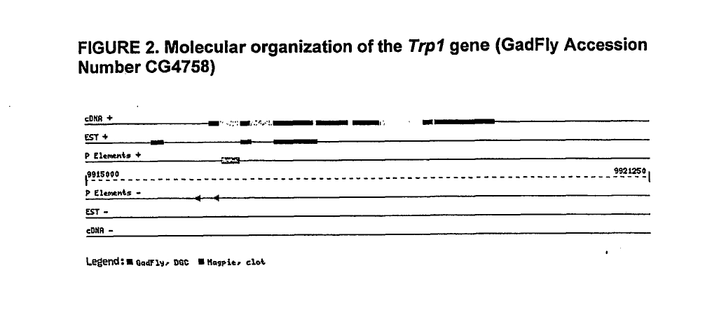

Using those isolated genomic sequences public databases like Berkeley Drosophila Genome Project (GadFly) were screened thereby identifying the integration site of the vectors, and the corresponding genes, described in more detail in the EXAMPLES section. The molecular organization of the genes is shown in FIGS. 2, 5, and 10, respectively.

The invention also encompasses polynucleotides that encode Trp1, MCT, or Ftz-F1 and homologous proteins. Accordingly, any nucleic acid sequence, which encodes the amino acid sequences of Trp1, MCT, or Ftz-F1 homologous proteins can be used to generate recombinant nucleic acid molecules that express Trp1, MCT, or Ftz-F1 homologous proteins. In a particular embodiment, the invention encompasses a nucleic acid sequence encoding Trp1 (GadFly Accession Number CG4758; GenBank Accession Number Z38100 for the cDNA, SPTREMBL Accession Number Q24559 for the protein), the human translocation protein 1 (GenBank Accession Number NM_{—}003262 for the cDNA, NP_{—}003253 for the protein), MCT (GadFly Accession Number CG8051, SEQ ID NO: 1 for the cDNA, SEQ ID NO: 2 for the protein), the human monocarboxylate transporter 3 (MCT3; GenBank Accession Number NM_{—}013356 for the cDNA, NP_{—}037488 for the protein) the human solute carrier family 16-monocarboxylic acid transporters-member 5 (GenBank Accession Number NM_{—}004695.2 for the cDNA, NP_{—}004686 for the protein), or Ftz-F1 (GenBank Accession Number M63711 for Ftz-F1 alpha, Accession Number M98397 for Ftz-F1 beta), or the human Ftz-F1 homologous nucleic acids, particularly nucleic acids encoding human members of the nuclear receptor subfamily (GenBank Accession Number NM_{—}003822; derived from GenBank Accession Number AF146343; GenBank Accession Number NM_{—}004959, derived from GenBank Accession Number U76388) It will be appreciated by those skilled in the art that as a result of the degeneracy of the genetic code, a multitude of nucleotide sequences encoding Trp1, MCT, or Ftz-F1 homologous proteins, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequences of naturally occurring Trp1, MCT, or Ftz-F1 homologous proteins, and all such variations are to be considered as being specifically disclosed. Codons may be selected to increase the rate at which expression of the peptide occurs in a particular prokaryotic or eukaryotic host in accordance with the frequency with which particular codons are utilised by the host. Other reasons for substantially altering the nucleotide sequence encoding Trp1, MCT, or Ftz-F1 homologous proteins and its derivatives without altering the encoded amino acid sequences include the production of RNA transcripts having more desirable properties, such as a greater half-life, than transcripts produced from the naturally occurring sequences. The invention also encompasses production of DNA sequences, or portions thereof, which encode Trp1, MCT, or Ftz-F1 homologous proteins and its derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding Trp1, MCT, or Ftz-F1 homologous proteins any portion thereof.

Also encompassed by the invention are polynucleotide sequences that are capable of hybridising to the claimed nucleotide sequences, and in particular, those shown in GenBank Acc. No. Z38100, SEQ ID NO: 1 (GadFly Accession Number CG8051), GenBank Acc. No. M63711, or GenBank Acc. No. M98397 or their human homologues under various conditions of stringency. Hybridisation conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding Trp1, MCT, or Ftz-F1 homologous proteins which are encompassed by the invention include deletions, insertions, or substitutions of different nucleotides resulting in a polynucleotide that encodes the same or a functionally equivalent Trp1, MCT, or Ftz-F1 homologous proteins.

The encoded proteins may also contain deletions, insertions, or substitutions of amino acid residues, which produce a silent change and result in a functionally equivalent Trp1, MCT, or Ftz-F1 homologous proteins. Deliberate amino acid substitutions may be made on the basis of similarity in polarity, charge, solubility, hydrophobicity, hydrophilicity, and/or the amphipathic nature of the residues as long as the biological activity of Trp1, MCT, or Ftz-F1 homologous proteins is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding Trp1, MCT, or. Ftz-F1 homologous proteins. As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence. Methods for DNA sequencing which are well known and generally available in the art may be used to practice any embodiments of the invention. The nucleic acid sequences encoding Trp1, MCT, or Ftz-F1 homologous proteins may be extended utilising a partial nucleotide sequence and employing various methods known in the art to detect upstream sequences such as promoters and regulatory elements.

When screening for full-length cDNAs, it is preferable to use libraries that have been size-selected to include larger cDNAs. Also, random-primed libraries are preferable, in that they will contain more sequences, which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions. Capillary electrophoresis systems, which are commercially available, may be used to analyse the size or confirm the nucleotide sequence of sequencing or PCR products.

In another embodiment of the invention, polynucleotide sequences or fragments thereof which encode Trp1, MCT, or Ftz-F1 homologous proteins, or fusion proteins or functional equivalents thereof, may be used in recombinant DNA molecules to direct expression of Trp1, MCT, or Ftz-F1 homologous proteins in appropriate host cells. Due to the inherent degeneracy of the genetic code, other DNA sequences, which encode substantially the same, or a functionally equivalent amino acid sequence may be produced and these sequences may be used to clone and express Trp1, MCT, or Ftz-F1 homologous proteins. As will be understood by those of skill in the art, it may be advantageous to produce Trp1, MCT, or Ftz-F1 homologous protein-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. The nucleotide sequences of the present invention can be engineered using methods generally known in the art in order to alter Trp1, MCT, or Ftz-F1 homologous proteins encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and, so forth.

In another embodiment of the invention, natural, modified, or recombinant nucleic acid sequences encoding Trp1, MCT, or Ftz-F1 homologous proteins may be ligated to a heterologous sequence to encode a fusion protein. For example, to screen peptide libraries for inhibitors of Trp1, MCT, or Ftz-F1 homologous proteins activities, it may be useful to encode chimerical Trp1, MCT, or Ftz-F1 homologous proteins proteins that can be recognised by commercially available antibodies. A fusion protein may also be engineered to contain a cleavage site located between the Trp1, MCT, or Ftz-F1 homologous proteins encoding sequence and the heterologous protein sequences, so that Trp1, MCT, or Ftz-F1 homologous proteins may be cleaved and purified away from the heterologous moiety. In another embodiment, sequences encoding Trp1, MCT, or Ftz-F1 homologous proteins may be synthesised, in whole or in part, using chemical methods well known in the art (see Caruthers, M. H. et al. (1980) Nucl. Acids Res. Symp. Ser. 7:215-223, Horn, T. et al. (1980) Nucl. Acids Res. Symp. Ser. 7:225-232). Alternatively, the proteins themselves may be produced using chemical methods to synthesise the amino acid sequence of Trp1, MCT, or Ftz-F1 homologous proteins, or a portion thereof. For example, peptide synthesis can be performed using various solid-phase techniques (Roberge, J. Y. et al. (1995) Science 269:202-204) and automated synthesis may be achieved, for example, using the ABI 431A peptide synthesiser (Perkin Elmer). The newly synthesised peptide may be substantially purified by preparative high performance liquid chromatography (e.g., Creighton, T. (1983) Proteins, Structures and Molecular Principles, WH Freeman and Co., New York, N.Y.). The composition of the synthetic peptides may be confirmed by amino acid analysis or sequencing (e.g., the Edman degradation procedure; Creighton, supra). Additionally, the amino acid sequences of Trp1, MCT, or Ftz-F1 homologous proteins, or any part thereof, may be altered during direct synthesis and/or combined using chemical methods with sequences from other proteins, or any part thereof, to produce a variant polypeptide.

In order to express a biologically active Trp1, MCT, or Ftz-F1 homologous proteins, the nucleotide sequences encoding Trp1, MCT, or Ftz-F1 homologous proteins functional equivalents, may be inserted into appropriate expression vectors, i.e., a vector, which contains the necessary elements for the transcription and translation of the inserted coding sequence. Methods, which are well known to those skilled in the art, may be used to construct expression vectors containing sequences encoding Trp1, MCT, or Ftz-F1 homologous proteins and appropriate transcriptional and translational control elements. These methods include in vitro recombinant DNA techniques. synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilised to contain and express sequences encoding Trp1, MCT, or Ftz-F1 homologous proteins. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems. The “control elements” or “regulatory sequences” are those non-translated regions of the vector-enhancers, promoters, 5′ and 3′ untranslated regions which interact with host cellular proteins to carry out transcription and translation. Such elements may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPorT1 plasmid (Gibco BRL) and the like may be used. The baculovirus polyhedrin promoter may be used in insect cells. Promoters and enhancers derived from the genomes of plant cells (e.g., heat shock, RUBISCO; and storage protein genes) or from plant viruses (e.g., viral promoters and leader sequences) may be cloned into the vector. In mammalian cell systems, promoters from mammalian genes or from mammalian viruses are preferable. If it is necessary to generate a cell line that contains multiple copies of the sequences encoding Trp1, MCT, or Ftz-F1 homologous proteins, vectors based on SV40 or EBV may be used with an appropriate selectable marker.

In bacterial systems, a number of expression vectors may be selected depending upon the use intended for Trp1, MCT, or Ftz-F1 homologous proteins. For example, when large quantities of Trp1, MCT, or Ftz-F1 homologous proteins are needed for the induction of antibodies, vectors, which direct high level expression of fusion proteins that are readily purified, may be used. Such vectors include, but are not limited to, the multifunctional E. coli cloning and expression vectors such as the BLUESCRIPT phagemid (Stratagene), in which the sequence encoding Trp1, MCT, or Ftz-F1 homologous proteins may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. PGEX vectors (Promega, Madison, Wis.) may also be used to express foreign polypeptides as fusion proteins with Glutathione S-Transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will. In the yeast, Saccharomyces cerevisiae, a number of vectors containing constitutive or inducible promoters such as alpha factor, alcohol oxidase, and PGH may be used. For reviews, see Ausubel et al., (supra) and Grant et al. (1987) Methods Enzymol. 153:516-544.

In cases where plant expression vectors are used, the expression of sequences encoding Trp1, MCT, or Ftz-F1 homologous proteins may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express Trp1, MCT, or Ftz-F1 homologous proteins. For example, in one such system, Autographa californica nuclear polyhedrosis virus (AcNPV) is used as a vector to express foreign genes in Spodoptera frugiperda cells or in Trichoplusia larvae. The sequences encoding Trp1, MCT, or Ftz-F1 homologous proteins may be cloned into a non-essential region of the virus, such as the polyhedrin gene, and place under control of the polyhedrin promoter. Successful insertions of Trp1, MCT, or Ftz-F1 homologous proteins will render the polyhedrin gene inactive and produce recombinant virus lacking coat protein. The recombinant viruses may then be used to infect, for example, S. frugiperda cells of Trichoplusia larvae in which Trp1, MCT, or Ftz-F1 homologous proteins may be expressed (Engelhard, E. K. et al. (1994) Proc. Nat. Acad. Sci. 91:3224-3227).

In mammalian host cells, a number of viral-based expression systems may be utilised. In cases where an adenovirus is used as an expression vector, sequences encoding Trp1, MCT, or Ftz-F1 homologous proteins may be ligated into an adenovirus transcription/translation complex consisting of the late promoter and tripartite leader sequence. Insertion in a non-essential E1 or E3 region of the viral genome may be used to obtain viable viruses which are capable of expressing Trp1, MCT, or Ftz-F1 homologous proteins in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding Trp1, MCT, or Ftz-F1 homologous proteins. Such signals include the ATG initiation codon and adjacent sequences. In cases where sequences encoding Trp1, MCT, or Ftz-F1 homologous proteins, its initiation codons, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express Trp1, MCT, or Ftz-F1 homologous proteins may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells, which successfully express the introduced sequences. Resistant clones of stably transformed cells may be proliferated using tissue culture techniques appropriate to the cell type. Any number of selection systems may be used to recover transformed cell lines.

The presence of polynucleotide sequences encoding Trp1, MCT, or Ftz-F1 homologous proteins can be detected by DNA-DNA or DNA-RNA hybridisation or amplification using probes or portions or fragments of polynucleotides encoding Trp1, MCT, or Ftz-F1 homologous proteins. Nucleic acid amplification based assays involve the use of oligonucleotides or oligomers based on the sequences encoding Trp1, MCT, or Ftz-F1 homologous proteins to detect transformants containing DNA or RNA encoding Trp1, MCT, or Ftz-F1 homologous proteins. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of Trp1, MCT, or Ftz-F1 homologous proteins, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilising monoclonal antibodies reactive to two non-interfering epitopes on Trp1, MCT, or Ftz-F1 homologous proteins is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labelled hybridisation or PCR probes for detecting sequences related to polynucleotides encoding Trp1, MCT, or Ftz-F1 homologous proteins include oligo-labelling, nick translation, end-labelling or PCR amplification using a labelled nucleotide.

Alternatively, the sequences encoding Trp1, MCT, or Ftz-F1 homologous proteins, or any portions thereof may be cloned into a vector for the production of an mRNA probe. Such vectors are known in the art, are commercially available, and may be used to synthesise RNA probes in vitro by addition of an appropriate RNA polymerase such as T7, T3, or SP6 and labelled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding Trp1, MCT, or Ftz-F1 homologous proteins may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode Trp1, MCT, or Ftz-F1 homologous proteins may be designed to contain signal sequences, which direct secretion of Trp1, MCT, or Ftz-F1 homologous proteins through a prokaryotic or eukaryotic cell membrane. Other recombinant constructions may be used to join sequences encoding Trp1, MCT, or Ftz-F1 homologous proteins to nucleotide sequence encoding a polypeptide domain, which will facilitate purification of soluble proteins. Such purification facilitating domains include, but are not limited to, metal chelating peptides such as histidine-tryptophan modules that allow purification on immobilised metals, protein A domains that allow purification on immobilised immunoglobulin, and the domain utilised in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and Trp1, MCT, or Ftz-F1 homologous proteins may be used to facilitate purification. In addition to recombinant production, fragments of Trp1, MCT, or Ftz-F1 homologous proteins may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesiser (Perkin Elmer). Various fragments of Trp1, MCT, or Ftz-F1 homologous proteins may be chemically synthesised separately and combined using chemical methods to produce the full length molecule.

The nucleic acids encoding the proteins of the invention can be used to generate transgenic animal or site specific gene modifications in cell lines. Transgenic animals may be made through homologous recombination, where the normal locus of the genes encoding the proteins of the invention is altered. Alternatively, a nucleic acid construct is randomly integrated into the genome. Vectors for stable integration include plasmids, retrovirusses and other animal virusses, YACs, and the like. The modified cells or animal are useful in the study of the function and regulation of the proteins of the invention. For example, a series of small deletions and/or substitutions may be made in the genes that encode the proteins of the invention to determine the role of particular domains of the protein, functions in pancreatic differentiation, etc. Specific constructs of interest include anti-sense molecules, which will block the expression of the proteins of the invention, or expression of dominant negative mutations. A detectable marker, such as lac Z may be introduced in the locus of the genes of the invention, where upregulation of expression of the genes of the invention will result in an easily detected change in phenotype. One may also provide for expression of the genes of the invention or variants thereof in cells or tissues where it is not normally expressed or at abnormal times of development. In addition, by providing expression of the proteins of the invention in cells in which they are not normally produced, one can induce changes in cell behavior. DNA constructs for homologous recombination will comprise at least portions of the genes of the invention with the desired genetic modification, and will include regions of homology to the target locus. DNA constructs for random integration need not include regions of homology to mediate recombination. Conveniently, markers for positive and negative selection are included. Methods for generating cells having targeted gene modifications through homologous recombination are known in the art. For embryonic stem (ES) cells, an ES cell line may be employed, or embryonic cells may be obtained freshly from a host, e.g. mouse, rat, guinea pig etc. Such cells are grown on an appropriate fibroblast-feeder layer or grown in presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc. The transgenic animals may be used in functional studies, drug screening, etc.

Diagnostics and Therapeutics

The data disclosed in this invention show that the nucleic acids and proteins of the invention are useful in diagnostic and therapeutic applications implicated, for example but not limited to diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others. Hence, diagnostic and therapeutic uses for the Trp1, MCT, or Ftz-F1 homologous proteins proteins of the invention are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in diagnostic and therapeutic applications implicated in various diseases and disorders described below and/or other pathologies and disorders. For example, but not limited to, cDNAs encoding the Trp1, MCT, or Ftz-F1 homologous proteins proteins of the invention and particularly their human homologues may be useful in gene therapy, and the Trp1, MCT, or Ftz-F1 homologous proteins proteins of the invention and particularly their human homologues may be useful when administered to a subject in need thereof. By way of non-limiting example, the compositions of the present invention will have efficacy for treatment of patients suffering from diseases and disorders related to energy homeostasis, for example, but not limited to, metabolic diseases such as obesity, adipositas, eating disorders, wasting syndromes (cachexia), pancreatic dysfunctions (such as diabetes mellitus), hypertension, and other diseases such as arteriosclerosis, coronary artery disease (CAD), hypercholesterolemia, dyslipidemia, osteoarthritis, gallstones, cancer, e.g. cancers of the reproductive organs, sleep apnea, and others.

The nucleic acids encoding the Trp1, MCT, or Ftz-F1 homologous proteins of the invention, or fragments thereof, may further be useful in diagnostic applications, wherein the presence or amount of the nucleic acids or the proteins are to be assessed. These materials are further useful in the generation of antibodies that bind immunospecifically to the novel substances of the invention for use in therapeutic or diagnostic methods.

For example, in one aspect, antibodies which are specific for Trp1, MCT, or Ftz-F1 homologous proteins may be used directly as an antagonist, or indirectly as a targeting or delivery mechanism for bringing a pharmaceutical agent to cells or tissue which express Trp1, MCT, or Ftz-F1 homologous proteins. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunised by injection with Trp1, MCT, or Ftz-F1 homologous proteins, any fragment or oligopeptide thereof which has immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to Trp1, MCT, or Ftz-F1 homologous proteins have an amino acid sequence consisting of at least five amino acids, and more preferably at least 10 amino acids. It is preferable that they are identical to a portion of the amino acid sequence of the natural protein, and they may contain the entire amino acid sequence of a small, naturally occurring molecule. Short stretches of Trp1, MCT, or Ftz-F1 amino acids may be fused with those of another protein such as keyhole limpet hemocyanin and antibody produced against the chimeric molecule.

Monoclonal antibodies to Trp1, MCT, or Ftz-F1 and homologous proteins may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce Trp1, MCT, or Ftz-F1 homologous proteins- and -specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for Trp1, MCT, or Ftz-F1 homologous proteins, may also be generated. For example, such fragments include, but are not limited to, the F(ab′)2 fragments which can be produced by Pepsin digestion of the antibody molecule and the Fab fragments which can be generated by reducing the disulfide bridges of F(ab′)2 fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between Trp1, MCT, or Ftz-F1 homologous proteins and its specific antibody. A two-site, monoclonal-based immunoassay utilising monoclonal antibodies reactive to two non-interfering Trp1, MCT, or Ftz-F1 homologous proteins epitopes is preferred, but a competitive binding assay may also be employed (Maddox, supra).

In another embodiment of the invention, the polynucleotides encoding Trp1, MCT, or Ftz-F1 homologous proteins, or any fragment thereof, or antisense molecules, may be used for therapeutic purposes. In one aspect, antisense to the polynucleotide encoding Trp 1, MCT, or Ftz-F1 homologous proteins may be used in situations in which it would be desirable to block the transcription of the mRNA. In particular, cells may be transformed with sequences complementary to polynucleotides encoding Trp1, MCT, or Ftz-F1 homologous proteins. Thus, antisense molecules may be used to modulate Trp1, MCT, or Ftz-F1 homologous proteins activity, or to achieve regulation of gene function. Such technology is now well know in the art, and sense or antisense oligomers or larger fragments, can be designed from various locations along the coding or control regions of sequences encoding Trp1, MCT, or Ftz-F1 homologous proteins. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the gene encoding Trp1, MCT, or Ftz-F1 homologous proteins. These techniques are described both in Sambrook et al. (supra) and in Ausubel et al. (supra). Genes encoding Trp1, MCT, or Ftz-F1 homologous proteins can be turned off by transforming a cell or tissue with expression vectors which express high levels of polynucleotide or fragment thereof which encodes Trp1, MCT, or Ftz-F1 homologous proteins. Such constructs may be used to introduce untranslatable sense or antisense sequences into a cell. Even in the absence of integration into the DNA, such vectors may continue to transcribe RNA molecules until they are disabled by endogenous nucleases.

Transient expression may last for a month or more with a non-replicating vector and even longer if appropriate replication elements are part of the vector system.

As mentioned above, modifications of gene expression can be obtained by designing antisense molecules, DNA, RNA, or PNA, to the control regions of the gene encoding Trp1, MCT, or Ftz-F1 homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding Trp1, MCT, or Ftz-F1 homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Antisense molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding Trp1, MCT, or Ftz-F1 homologous proteins. Such DNA sequences may be incorporated into a variety of vectors with suitable RNA polymerase promoters such as T7 or SP6. Alternatively, these cDNA constructs that synthesise antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above.

Such pharmaceutical compositions may consist of Trp1, MCT, or Ftz-F1 homologous proteins, antibodies to Trp1, MCT, or Ftz-F1 homologous proteins, mimetics, agonists, antagonists, or inhibitors of Trp1, MCT, or Ftz-F1 homologous proteins. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping and/or lyophilising processes.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example Trp1, MCT, or Ftz-F1 homologous proteins fragments thereof, antibodies of Trp1, MCT, or Ftz-F1 homologous proteins, which is efficient for the treatment of a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combinations, reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind Trp1, MCT, or Ftz-F1 homologous proteins may be used for the diagnosis of conditions or diseases characterised by or associated with over- or underexpression of Trp1, MCT, or Ftz-F1 homologous proteins, or in assays to monitor patients being treated with Trp1, MCT, or Ftz-F1 homologous proteins, agonists, antagonists or inhibitors. The antibodies useful for diagnostic purposes may be prepared in the same manner as those described above for therapeutics. Diagnostic assays for Trp1, MCT, or Ftz-F1 homologous proteins include methods, which utilise the antibody and a label to detect Trp1, MCT, or Ftz-F1 homologous proteins in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring Trp1, MCT, or Ftz-F1 homologous proteins are known in the art and provide a basis for diagnosing altered or abnormal levels of Trp1, MCT, or Ftz-F1 homologous proteins expression. Normal or standard values for Trp1, MCT, or Ftz-F1 homologous proteins expression are established by combining body fluids or cell extracts taken from normal mammalian subjects, preferably human, with antibody to Trp1, MCT, or Ftz-F1 homologous proteins under conditions suitable for complex formation. The amount of standard complex formation may be quantified by various methods, but preferably by photometry, means. Quantities of Trp1, MCT, or Ftz-F1 homologous proteins expressed in control and disease, samples from biopsied tissues are compared with the standard values. Deviation between standard and subject values establishes the parameters for diagnosing disease.

In another embodiment of the invention, the polynucleotides encoding Trp1, MCT, or Ftz-F1 homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of Trp1, MCT, or Ftz-F1 homologous proteins may be correlated with disease. The diagnostic assay may be used to distinguish between absence, presence, and excess expression of Trp1, MCT, or Ftz-F1 homologous proteins, and to monitor regulation of Trp1, MCT, or Ftz-F1 homologous proteins levels during therapeutic intervention.

In one aspect, hybridisation with PCR probes which are capable of detecting polynucleotide sequences, including genomic sequences, encoding Trp1, MCT, or Ftz-F1 homologous proteins closely related molecules, may be used to identify nucleic acid sequences which encode Trp1, MCT, or Ftz-F1 homologous proteins. The specificity of the probe, whether it is made from a highly specific region, e.g., unique nucleotides in the 5′ regulatory region, or a less specific region, e.g., especially in the 3′ coding region, and the stringency of the hybridisation or amplification (maximal, high, intermediate, or low) will determine whether the probe identifies only naturally occurring sequences encoding Trp1, MCT, or Ftz-F1 homologous proteins, alleles, or related sequences. Probes may also be used for the detection of related sequences, and should preferably contain at least 50% of the nucleotides from any of the Trp1, MCT, or Ftz-F1 homologous proteins encoding sequences. The hybridisation probes of the subject invention may be DNA or RNA and derived from the nucleotide sequence of GenBank Acc. No. Z38100, SEQ ID NO: 1, GenBank Acc. No. M63711, or GenBank Acc. No. M98397, or from a human homologous sequence thereof or from a genomic sequence including promoter, enhancer elements, and introns of the naturally occurring Trp1, MCT, or Ftz-F1 homologous proteins. Means for producing specific hybridisation probes for DNAs encoding Trp1, MCT, or Ftz-F1 homologous proteins include the cloning of nucleic acid sequences encoding Trp1, MCT, or Ftz-F1 homologous proteins derivatives into vectors for the production of mRNA probes. Such vectors are known in the art, commercially available, and may be used to synthesise RNA probes in vitro by means of the addition of the appropriate RNA polymerases and the appropriate labelled nucleotides. Hybridisation probes may be labelled by a variety of reporter groups, for example, radionuclides such as 32P or 35S, or enzymatic labels, such as alkaline phosphatase coupled to the probe via avidin/biotin coupling systems, and the like.

Polynucleotide sequences encoding Trp1, MCT, or Ftz-F1 homologous proteins may be used for the diagnosis of conditions or diseases, which are associated with expression of Trp1, MCT, or Ftz-F1 homologous proteins. Examples of such conditions or diseases include, but are not limited to, pancreatic diseases and disorders, including diabetes. Polynucleotide sequences encoding Trp1, MCT, or Ftz-F1 homologous proteins may also be used to monitor the progress of patients receiving treatment for pancreatic diseases and disorders, including diabetes. The polynucleotide sequences encoding Trp1, MCT, or Ftz-F1 homologous proteins may be used in Southern or Northern analysis, dot blot, or other membrane-based technologies; in PCR technologies; or in dip stick, pin, ELISA or chip assays utilising fluids or tissues from patient biopsies to detect altered Trp1, MCT, or Ftz-F1 homologous proteins expression. Such qualitative or quantitative methods are well known in the art.

In a particular aspect, the nucleotide sequences encoding Trp1, MCT, or Ftz-F1 homologous proteins may be useful in assays that detect activation or induction of various metabolic diseases and disorders, including diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others. The nucleotide sequences encoding Trp1, MCT, or Ftz-F1 homologous proteins may be labelled by standard methods, and added to a fluid or tissue sample from a patient under conditions suitable for the formation of hybridisation complexes. After a suitable incubation period, the sample is washed and the signal is quantitated and compared with a standard value. If the amount of signal in the biopsied or extracted sample is significantly altered from that of a comparable have hybridised with nucleotide sequences in the sample, and the presence of altered levels of nucleotide sequences encoding Trp1, MCT, or Ftz-F1 homologous proteins in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of Trp1, MCT, or Ftz-F1 homologous proteins, a normal or standard profile for expression is established. This may be accomplished by combining body fluids or cell extracts taken from normal subjects, either animal or human, with a sequence, or a fragment thereof, which encodes Trp1, MCT, or Ftz-F1 homologous proteins, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases and disorders as described above, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders. Additional diagnostic uses for oligonucleotides designed from the sequences encoding Trp1, MCT, or Ftz-F1 homologous proteins may involve the use of PCR. Such oligomers may be chemically synthesised, generated enzymatically, or produced from a recombinant source. Oligomers will preferably consist of two nucleotide sequences, one with sense orientation (5′.fwdarw.3′) and another with antisense (3′.rarw.5′), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of Trp1, MCT, or Ftz-F1 homologous proteins include radiolabelling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or colorimetric response gives rapid quantification.

In another embodiment of the invention, the nucleic acid Trp1, MCT, or Ftz-F1 homologous proteins sequences, which encode Trp1, MCT, or Ftz-F1 homologous proteins, may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding Trp1, MCT, or Ftz-F1 homologous proteins on a physical chromosomal map and a specific disease, or predisposition to a specific disease, may help to delimit the region of DNA associated with that genetic disease.

The nucleotide sequences of the subject invention may be used to detect differences in gene sequences between normal, carrier, or affected individuals. In situ hybridisation of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localised by genetic linkage to a particular genomic region, for example, AT to 11 q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, the proteins of the invention, their catalytic or immunogenic fragments or oligopeptides thereof, an in vitro model, a genetically altered cell or animal, can be used for screening libraries of compounds in any of a variety of drug screening techniques. One can identify ligands or substrates that bind to, modulate or mimic the action of one or more of the proteins of the invention. The fragment employed in such screening may be free in solution, affixed to a solid support, borne on a cell surface, or located intracellularly. The formation of binding complexes, between the proteins of the invention and the agent tested, may be measured. Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to Trp1, MCT, or Ftz-F1 homologous proteins large numbers of different small test compounds are provided or synthesised on a solid substrate, such as plastic pins or some other surface. The test compounds are reacted with Trp1, MCT, or Ftz-F1 homologous proteins, or fragments thereof, and washed. Bound Trp1, MCT, or Ftz-F1 homologous proteins are then detected by methods well known in the art. Purified Trp1, MCT, or Ftz-F1 homologous proteins can also be coated directly onto plates for use in the aforementioned drug screening techniques. Alternatively, non-neutralising antibodies can be used to capture the peptide and immobilise it on a solid support. In another embodiment, one may use competitive drug screening assays in which neutralising antibodies capable of binding Trp1, MCT, or Ftz-F1 homologous proteins specifically compete with a test compound for binding Trp1, MCT, or Ftz-F1 homologous proteins. In this manner, the antibodies can be used to detect the presence of any peptide, which shares one or more antigenic determinants with Trp1, MCT, or Ftz-F1 homologous proteins. In additional embodiments, the nucleotide sequences which encode Trp1, MCT, or Ftz-F1 homologous proteins may be used in any molecular biology techniques that have yet to be developed, provided the new techniques rely on properties of nucleotide that are currently known, including, but not limited to, such properties as the triplet genetic code and specific base pair interactions.

Finally, the invention relates to kits comprising nucleic acids, proteins and effector molecules as described above.

The Figures show:

FIG. 1 shows the decrease of triglyceride content of EP(2)0663 flies caused by heterozygous integration of the P-vector (in comparison to controls).

FIG. 2 shows the molecular organisation of the Trp1 gene locus. The dark grey boxes on line ‘cDNA +’ shows the CG4758 prediction; the boxes on line ‘EST+’ (left to right) represent Clot 896_{—}2 and two symbols for Clot 896_{—}1; and the box on the line ‘P Elements +’ refers to the I(2)k13305 P-vector integration which causes also a decrease in triglyceride content; and the arrows on line ‘P Elements −’ show the location of the EP(2)0663 integration.

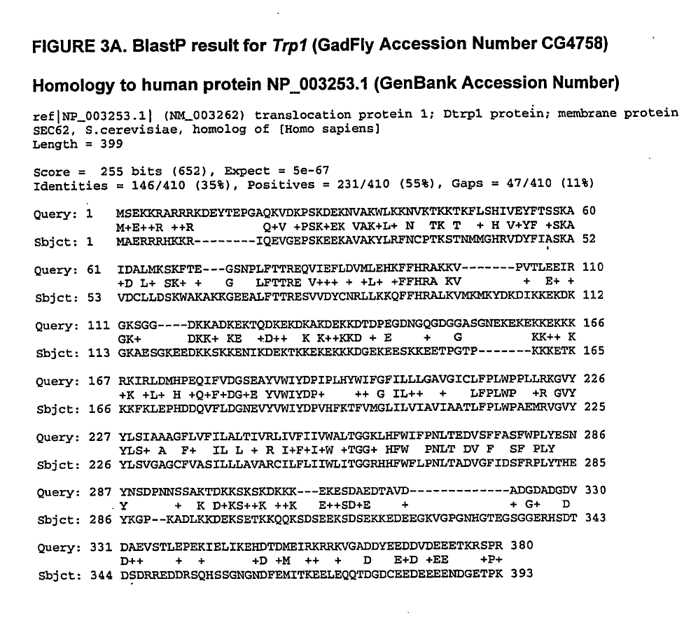

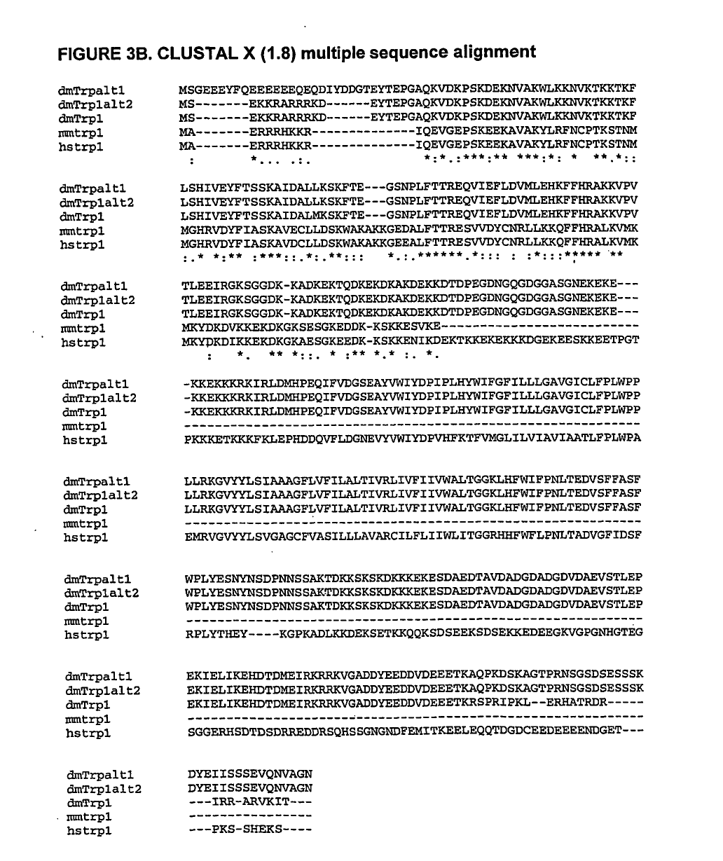

FIG. 3A shows the BlastP result for Trp1. Shown is the Alignment with the best human match.

FIG. 3B shows the comparison (CLUSTAL X 1.8) of Drosophila isoforms (dmTrpalt1, Acc. No. AAF52847; dmTrpalt2, Acc. No. AAF52848; dmTrp1, Acc. No. Q24559), mouse (mmtrp1, Acc. No. BAB29058), human (hstrp1, Acc. No. NP_{—}003253) homologue Trp1 proteins. Gaps in the alignment are represented as −.

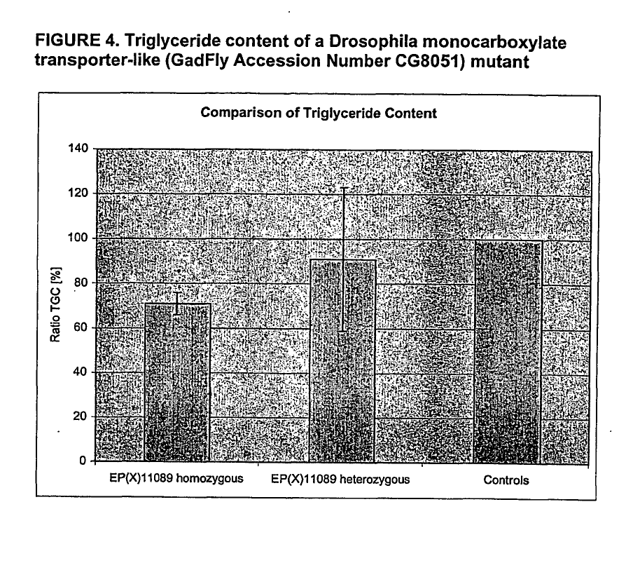

FIG. 4 shows the decrease of triglyceride content of EP(X)11089 flies caused by homozygous viable and heterozygous integration of the P-vector (in comparison to controls).

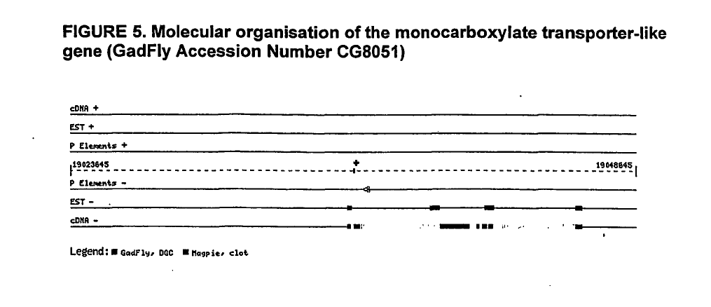

FIG. 5 shows the molecular organisation of the monocarboxylate transporter-like gene locus. The dark grey boxes on line ‘cDNA −’ shows the GadFly CG8051 prediction; the boxes on line ‘EST−’ (left to right) represent Clot 7515_{—}1 and DGC SD10554; and the ‘+’ symbol refers to the EP(X)11089 integration which causes a decrease in triglyceride content; and the arrow on line P Elements ‘−’ shows the location of the homozygous viable EP(X)1550 integration.

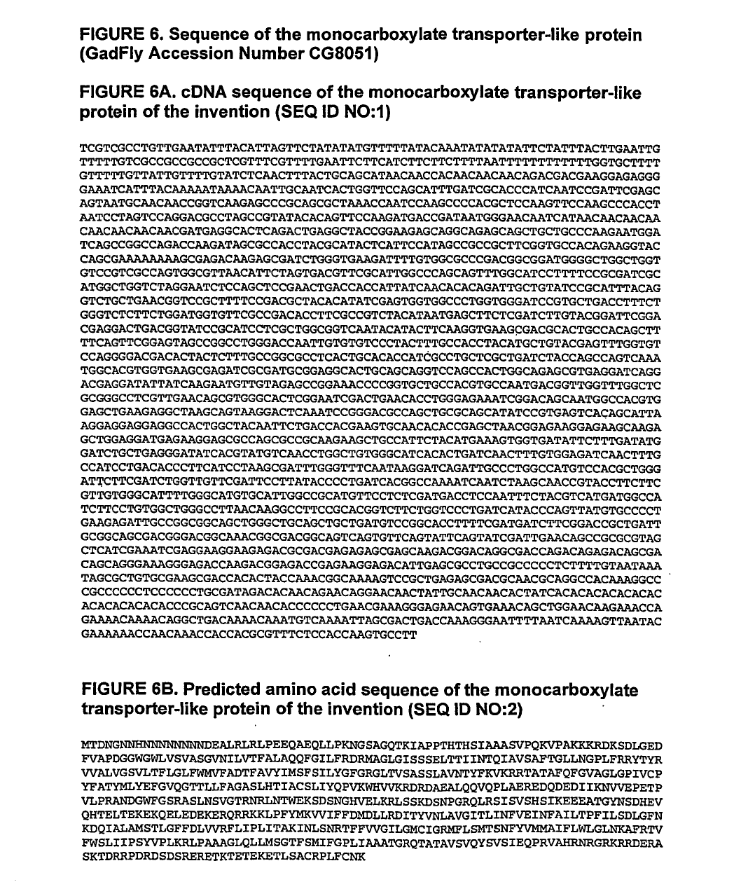

FIG. 6A shows the nucleic acid sequence of the most preferred gene of the invention (SEQ ID NO:1; CG8051)

FIG. 6B shows the most preferred protein sequence of the monocarboxylate transporter-like protein of the invention (SEQ ID NO:2; CG8051)

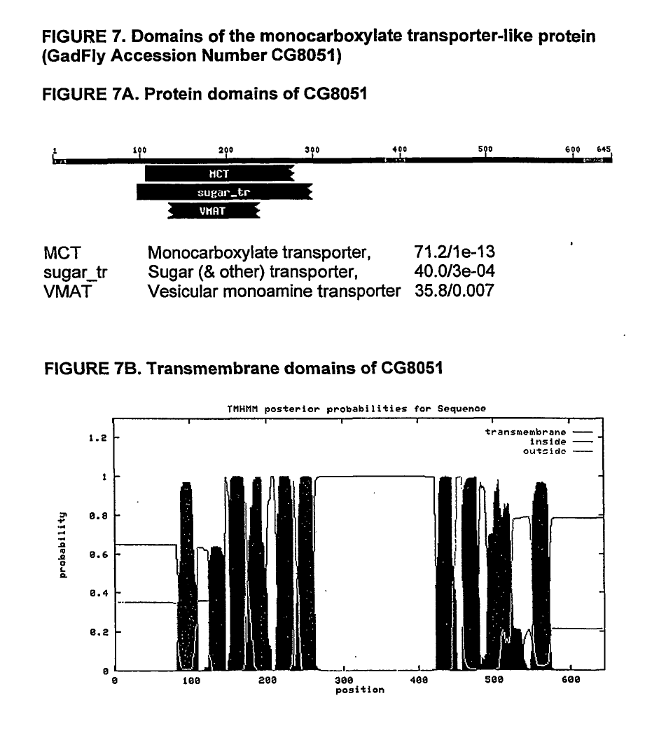

FIG. 7A shows protein domains of the monocarboxylate transporter-like protein of the invention. MCT refers to monocarboxylate transporter, sugar_tr refers to surgar transporter, and VMAT refers to vesicular monoamine transporter.

FIG. 7B shows a transmembrane domain plot of the MCT protein of the invention. The calculation was performed following J. Glasgow et al. Proc. Sixth Int. Conf. Of Intelligent Systems for Molecular Biology. 175-182-AAAI Press, 1998

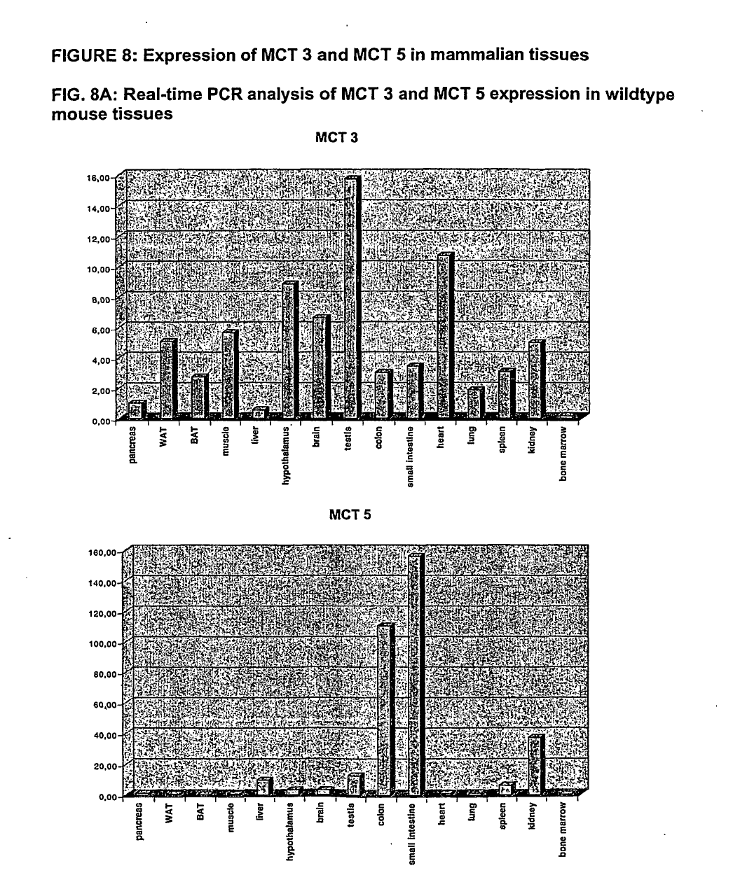

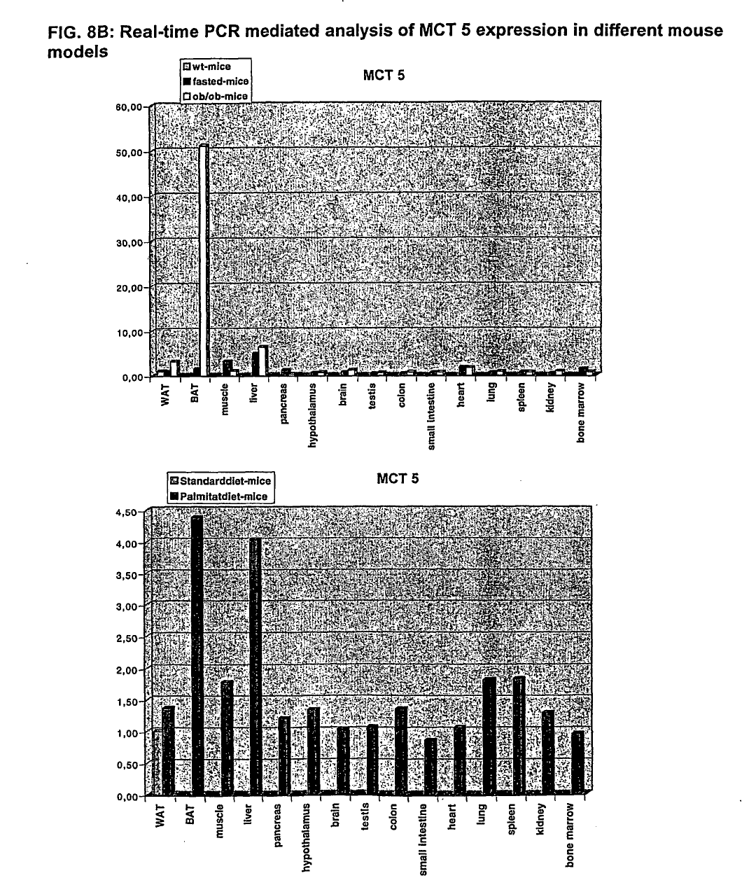

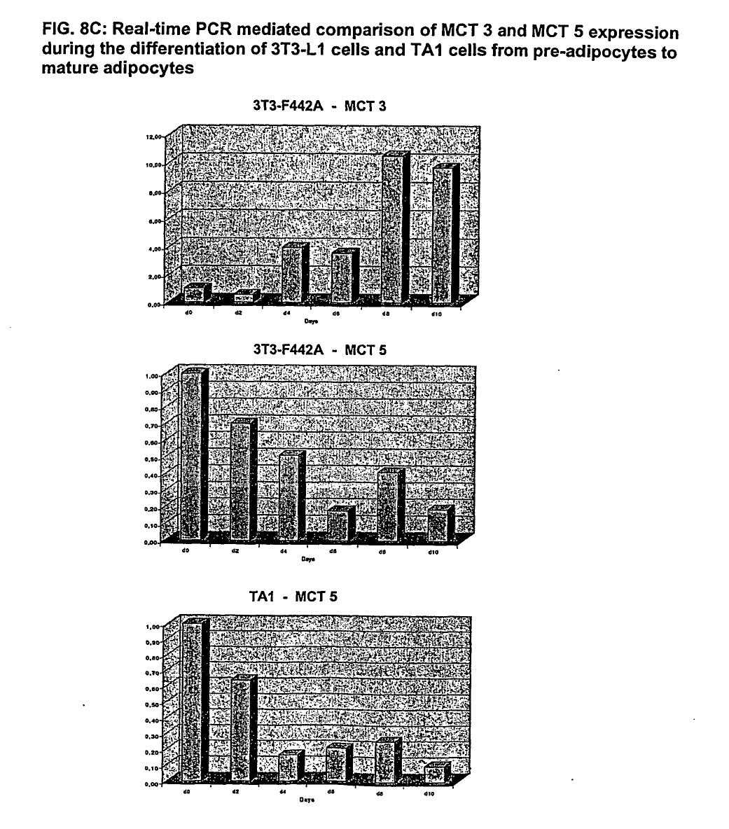

FIG. 8: Expression of MCT 3 and MCT 5 in mammalian tissues.

FIG. 8A shows the real-time PCR analysis of MCT 3 and MCT 5 expression in wildtype mouse tissues. The relative RNA-expression is shown on the left hand side, the tissues tested are given on the horizontal line (WAT=white adipose tissue, BAT=brown adipose tissue).

FIG. 8B shows the real-time PCR mediated analysis of MCT 5 expression in different mouse models. The relative RNA-expression is shown on the left hand side, the tissues tested are shown on the horizontal line (WAT=white adipose tissue, BAT=brown adipose tissue).

FIG. 8C shows the real-time PCR mediated comparison of MCT 3 and MCT 5 expression during the differentiation of 3T3-F442A cells and TA1 cells from preadipocytes to mature adipocytes. The relative RNA-expression is shown on the left hand side, the days of differention are shown on the horizontal line (d0=day 0, start of the experiment, until d10=day 10).

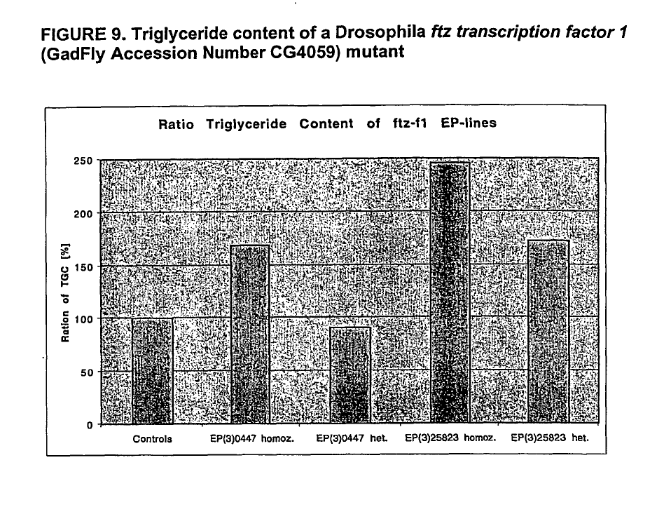

FIG. 9 shows the increase of triglyceride content of EP(3)0447 and EP(3)25823 flies caused by homozygous viable integration of the P-vector (in comparison to controls).

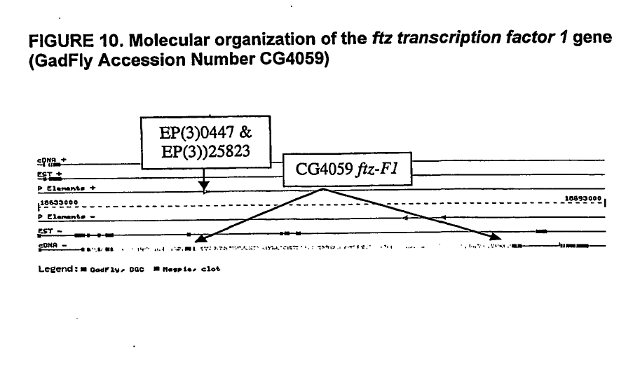

FIG. 10 shows the molecular organisation of the ftz-F1 gene locus.

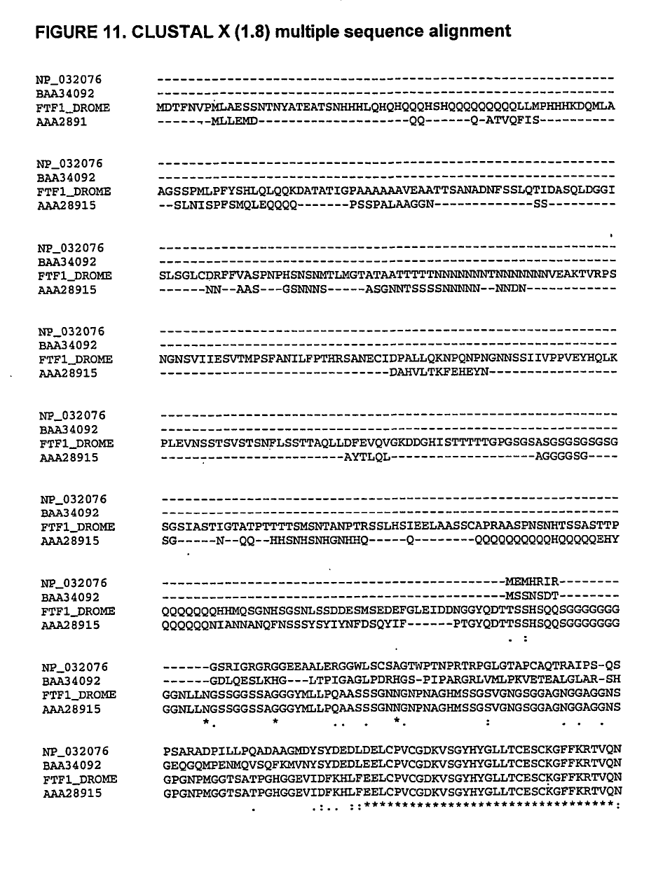

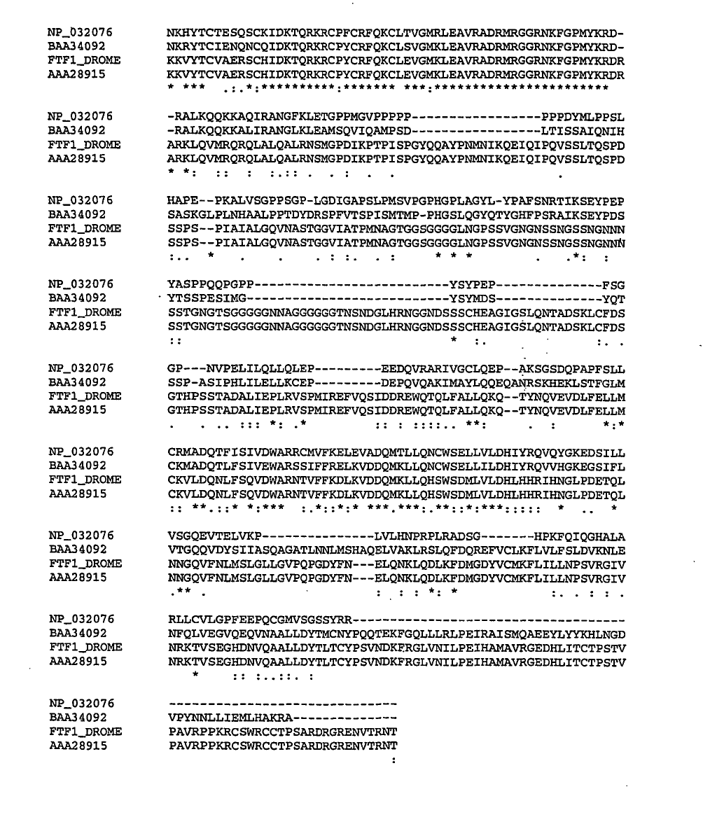

FIG. 11 shows the comparison (CLUSTAL X 1.8) of mouse (Acc. No. NP_{—}032076), human (Acc. No. BAA34092) and the two isoforms of Drosophila (Acc. No. AAA28542, Ftz-F1 alpha, here referred to as FTF1_DROME; and Acc. No. AAA28915, Ftz-F1 beta) homologue Ftz-F1 proteins. Gaps in the alignment are represented as −.

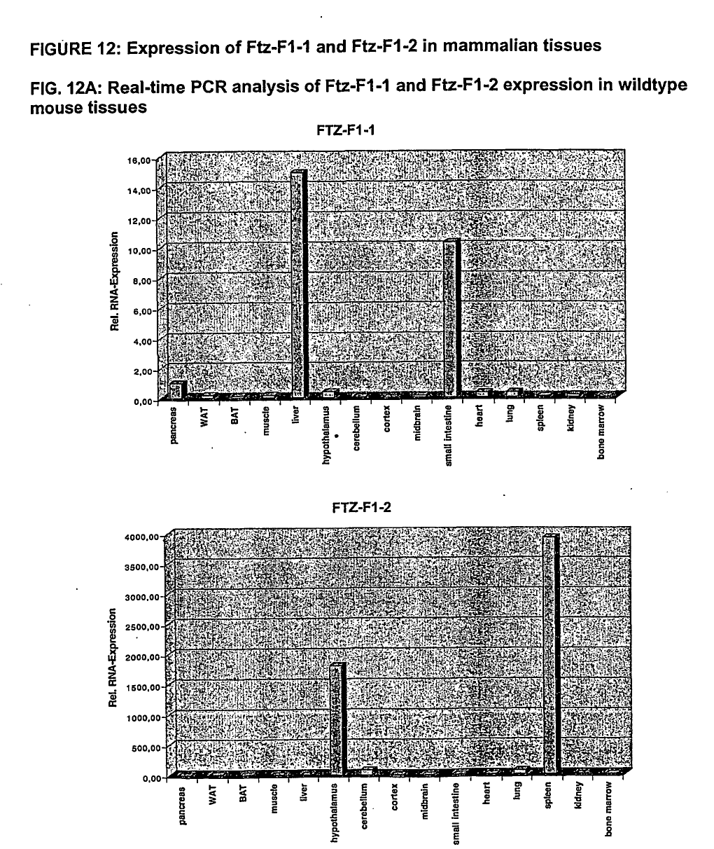

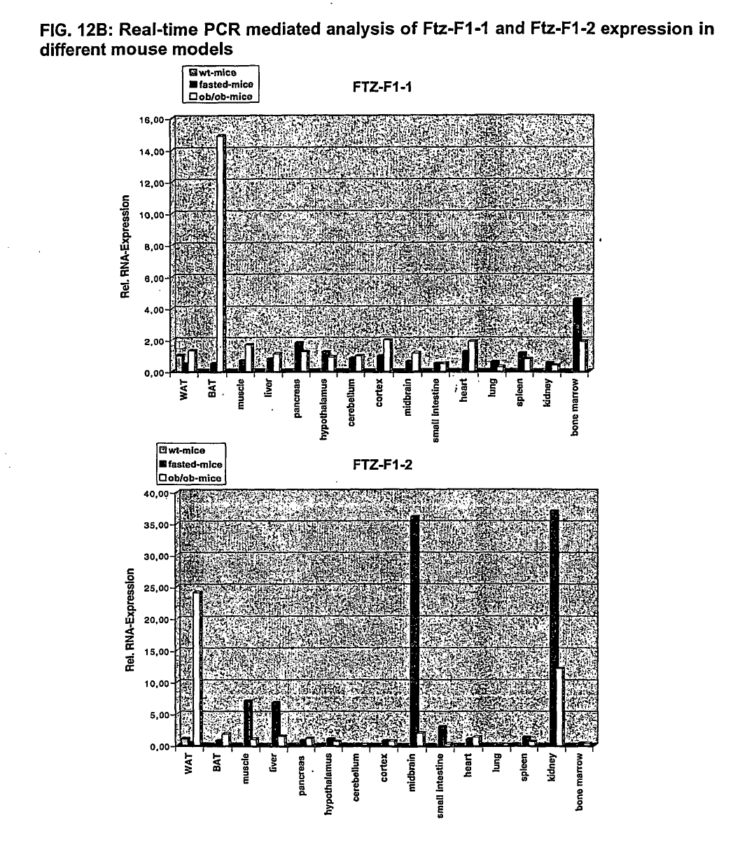

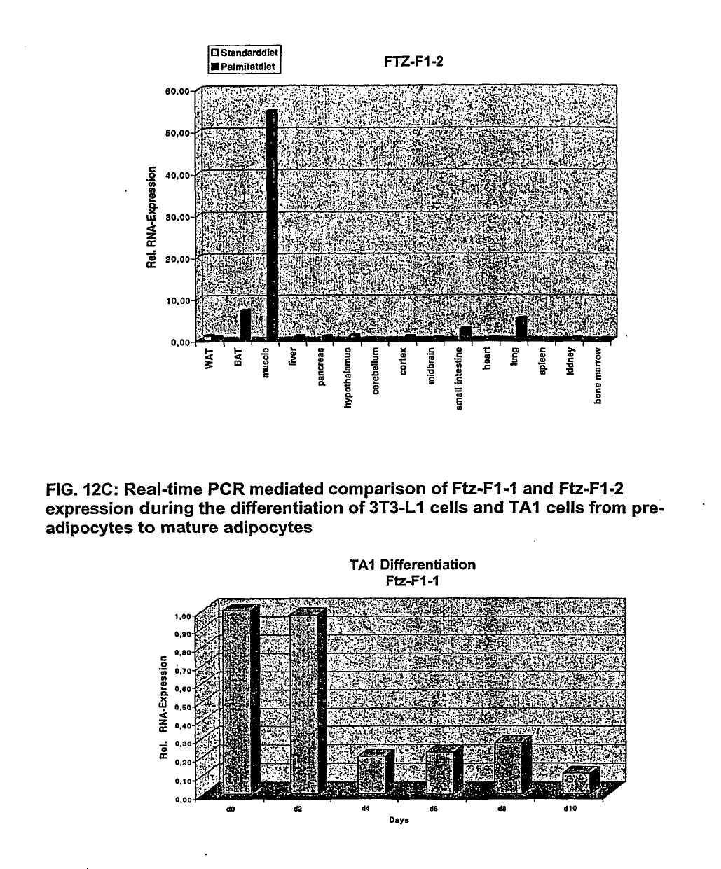

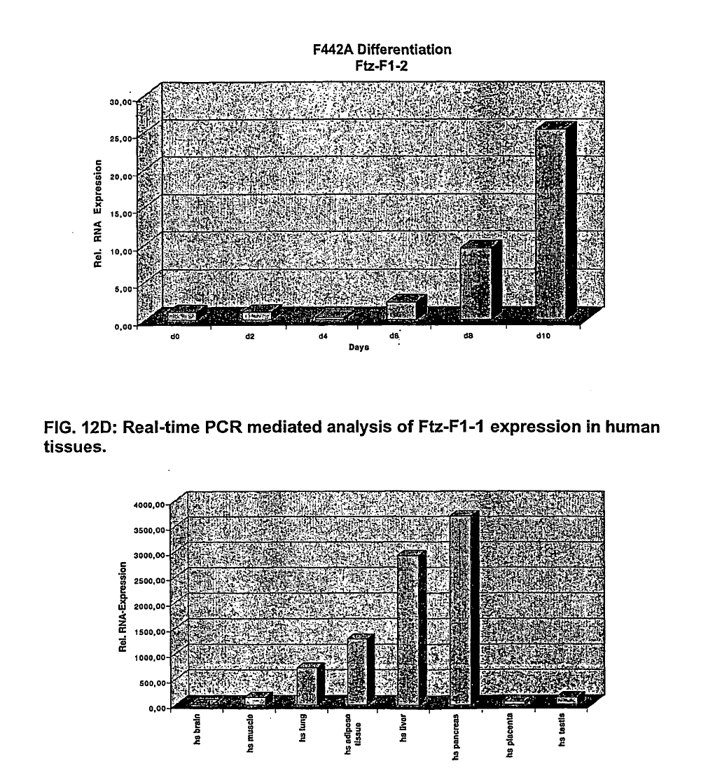

FIG. 12: Expression of Ftz-F1-1 and Ftz-F1-2 in mammalian tissues.

FIG. 12A shows the real-time PCR analysis of Ftz-F1-1 and Ftz-F1-2 expression in wildtype mouse tissues. The relative RNA-expression is shown on the left hand side, the tissues tested are given on the horizontal line (WAT=white adipose tissue, BAT=brown adipose tissue).

FIG. 12B shows the real-time PCR mediated analysis of Ftz-F1-1 and Ftz-F1-2 expression in different mouse models. The relative RNA-expression is shown on the left hand side, the tissues tested are shown on the horizontal line (WAT=white adipose tissue, BAT=brown adipose tissue).

FIG. 12C shows the real-time PCR mediated analysis of Ftz-F1-1 and Ftz-F1-2 expression during the differentiation of 3T3-F442A cells and TA1 cells from preadipocytes to mature adipocytes. The relative RNA-expression is shown on the left hand side, the days of differention are shown on the horizontal line (d0=day 0, start of the experiment, until d10=day 10).

FIG. 12D shows the real-time PCR mediated analysis of Ftz-F1-1 expression in human tissues.

The Examples illustrate the invention:

### EXAMPLE 1

### Measurement of Triglyceride Content

Males of the offspring of a cross or line were analyzed in a triglyceride assay. The average changes of triglyceride content of heterozygous EP(2)0663 flies, homozygous EP(X)11089 flies, and homozygous EP(3)0447 and EP(3)25823 flies were investigated in comparison to control flies (different wildtype populations) (FIGS. 1, 4, and 9). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assay was repeated several times. Wildtype flies show constantly a triglyceride level, which is shown as 100% in FIGS. 1, 4, and 9.

The result of the triglyceride content analysis of EP(2)0663 heterozygous flies is shown in FIG. 1. The average decrease of triglyceride content of the heterozygous EP(2)0663 is 60% (‘EP(2)0663 heterozygous’, FIG. 1, column 1), compared to control flies (different wildtype populations) (‘Controls’, FIG. 1, column 2). Therefore, the loss of gene activity in the chromosomal locus 2L, 30F5 (estimated), where the EP-vector of EP(2)0663 flies is semi-lethal integrated, is responsible for changes in the metabolism of the energy storage triglycerides, therefore representing in both cases an obese fly model. The decrease of triglyceride content due to the potential loss of a gene function suggests potential gene activities in energy homeostasis in a dose dependent manner that controls the amount of energy stored as triglycerides.

The result of the triglyceride content analysis of EP(X) 11089 homozygous flies is shown in FIG. 4. The average decrease of triglyceride content of the homozygous viable EP(X)11089 is 30% (‘EP(X)11089 homozygous’, FIG. 4, column 1). EP(X)11089 flies are shown in comparison to controls (different wildtype populations) (‘Controls’, FIG. 4, column 3) and to heterozygous EP(X) 11089 flies (‘EP(X) 11089 heterozygous’, FIG. 4, column 2). Therefore, the loss of gene activity in the chromosomal locus X, 18C1-2 (estimated), where the EP-vector of EP(X)11089 flies is viably integrated, is responsible for changes in the metabolism of the energy storage triglycerides, therefore representing in both cases an obese fly model. The decrease of triglyceride content due to the potential loss of a gene function suggests potential gene activities in energy homeostasis in a dose dependent manner that controls the amount of energy stored as triglycerides.

The result of the triglyceride content analysis of EP(3)0447 and EP(3)25823 homozygous flies is shown in FIG. 9. The average increase of triglyceride content of the homozygous viable EP(3)0447 is 69% (‘EP(3)0447 homoz.’, FIG. 9, column 2) and of the homozygous viable EP(3)25823 145% (‘EP(3)25823 homoz.’, FIG. 9, column 4). Even heterozygous EP(3)25823 flies show an increase of 72% of the triglyceride content (dosis effect) (‘EP(3)25823 het.’, FIG. 9, column 5). EP(3)25823 and EP(3)0477 flies are shown in comparison to controls (different wildtype populations) (‘Controls’, FIG. 9, column 1). Therefore, the very likely loss of gene activity in the gene locus 3L, 75D4-6 (estimated, chromosomal localisation where the EP-vector of EP(3)0447 and EP(3)25823 flies is homozygous viably integrated) is responsible for changes in the metabolism of the energy storage triglycerides, therefore representing in both cases an obese fly model, The increase of triglyceride content due to the potential loss of a gene function suggests potential gene activities in energy homeostasis in a dose dependent manner that controls the amount of energy stored as triglycerides.

### EXAMPLE 2

### Identificiation of the Drosophila Genes Associated with Energy Homeostasis and/or the Metabolism of Triglycerides

Nucleic acids encoding the Trp1 protein of the present invention were identified using plasmid-rescue technique. Genomic DNA sequences of about 0.8 kb were isolated that are localized directly 3′ to the EP(2)0663 integration. Using those isolated genomic sequences public databases like Berkeley Drosophila Genome Project (GadFly) were screened thereby confirming the integration site of EP(2)0663 nearby localized endogenous genes (FIG. 2). FIG. 2 shows the molecular organisation of the Trp1 locus. The genomic DNA sequence is represented by the assembly as a grey dotted line in the middle that includes the integration sites of EP(2)0663. Numbers represent the coordinates of the genomic DNA (starting at position 9915000 on chromosome 2L, ending at position 9921250). Transcribed DNA sequences (ESTs of DGC and clots) are shown as grey bars on the “EST +” line (ESTs from left to rigt:: clot 896_{—}2, two grey boxes for Clot 896_{—}1). Predicted genes are shown as grey bars on the “cDNA +” line (as predicted by GadFly & Magpie). Predicted exons of gene CG4758 (GadFly, Trp1) are shown as dark grey bars and introns as light grey bars on line ‘cDNA+’. Arrows on the “P-Elements−” line represent the EP-vector EP(2)0663 integration sites and the direction of ectopic expression of endogenous genes controlled by the Gal4 promoters in the EP-vectors.

EP(2)0663 is integrated into the intron of the Trp1 (CG4758) transcription unit in antisense orientation very close to each other. Trp1 is also represented by the ESTs Clot 896_{—}2 and Clot 896_{—}1. Clot 896_{—}2 and _{—}1 represent cDNA clones meaning that their DNA sequences are expressed in Drosophila. All EST sequences overlap with the sequence of the predicted gene CG4758 (Trp1) therefore EP(2)0663 are heterozygous semi-lethal integrated in the transcription unit of Trp 1. Expression of CG4758 could be effected by heterozygous semi-lethal integration of EP(2)0663 leading to decrease of the energy storage triglycerides.

Trp1 encodes for a gene that is predicted by GadFly sequence analysis programs (CG4758). No functional data described the regulation of obesity and metabolic diseases are available in the prior art for the genes with Acc. No. Z38100, AE003627, AE003627, and NM 003262, referred to as Trp 1 in the present invention.

Nucleic acids encoding the monocarboxylate transporter-like (MCT) protein of the present invention were identified using plasmid-rescue technique. Genomic DNA sequences of about 0.8 kb were isolated that are localized directly 3′ to the EP(X)11089 integration. Using those isolated genomic sequences public databases like Berkeley Drosophila Genome Project (GadFly) were screened thereby confirming the integration site of EP(X)11089 nearby localized endogenous genes (FIG. 5). FIG. 5 shows the molecular organisation of the MCT locus. The genomic DNA sequence is represented by the assembly as a black dotted line in the middle that includes the integration sites of EP(X) 11089 (grey arrow on line ‘P elements −’). Numbers represent the coordinates of the genomic DNA (starting at position 19023645 on chromosome X, ending at position 19048645). Transcribed DNA sequences (ESTs of DGC and clots) are shown as bars in the “EST −” line as grey bars (from left to right, clot 7515_{—}1, three grey boxes for DGC SD10554). Predicted genes are shown as grey bars on the “cDNA −” line (as predicted by GadFly & Magpie). Predicted exons of gene CG8051 (GadFly) are shown as dark grey bars and introns as light grey bars on line ‘cDNA−’. The arrow on the “P-Elements −” line represents the EP-vector EP(X)11089 integration site and the direction of ectopic expression of endogenous genes controlled by the Gal4 promoters in the EP-vectors.

EP(X)11089 is integrated into the intron of the CG8051 transcription unit in sense orientation very close to each other. Clot 7515_{—}1 and DGC SD10554 represent cDNA clones meaning that their DNA sequences are expressed in Drosophila. All EST sequences overlap with the sequence of the predicted gene CG8051 (MCT) therefore EP(X)11089 is homozygous viable integrated in the transcription unit of MCT. Expression of CG8051 could be effected by homozygous viable integration of EP(X) 11089 leading to decrease of the energy storage triglycerides.

The MCT protein of the invention is encoded by a gene of 2704 base pairs that is predicted by GadFly sequence analysis programs (CG8051). No functional data described the regulation of obesity and metabolic diseases are available in the prior art for the gene, referred to as MCT protein in the present invention.

The present invention is describing a polypeptide comprising the amino acid sequence of MCT protein, SEQ ID NO:2 (CGB051), as presented using the one-letter code in FIG. 6B. The MCT protein of the invention is 645 amino acids in length. An open reading was identified beginning with an ATP initiation codon at nucleotide 422 and ending with a stop codon at nucleotide 2356. The calculated molecular weight of the protein of the invention is 72139 dalton

Nucleic acids encoding the Ftz-F1 protein of the present invention were identified using plasmid-rescue technique. Genomic DNA sequences of about 0.8 kb were isolated that are localized directly 3′ to the EP(3)0447 and EP(3)25823 integration. Using those isolated genomic sequences public databases like Berkeley Drosophila Genome Project (GadFly) were screened thereby confirming the integration site of EP(3)0447 and EP(3)25823 nearby localized endogenous genes (FIG. 10). FIG. 10 shows the molecular organisation of the Ftz-F1 locus. The genomic DNA sequence is represented by the assembly as a black dotted line in the middle that includes the integration sites of EP(3)0447. Numbers represent the coordinates of the genomic DNA (starting at position 18633000 on chromosome 3L, ending at position 18693000). Transcribed DNA sequences (ESTs of DGC and clots) are shown as bars in the “EST −” line. Clot 3727_{—}2 and 1 and DGC LD34889 represent cDNA clones meaning that their DNA sequence are expressed in Drosophila. Predicted genes are shown as grey bars on the “cDNA −” line (as predicted by GadFly & Magpie). Predicted exons of gene CG4059 (GadFly, Ftz-F1) are shown as dark grey bars and introns as light grey bars. Grey arrows on the “P-Elements”-lines show the EP-vector integration sites. The overlapping arrows on the “P-Elements +” line represent the EP-vectors EP(3)0447 and EP(3)25823 integration sites and the direction of ectopic expression of endogenous genes controlled by the Gal4 promoters in the EP-vectors.

EP(3)0447 and EP(3)25823 are integrated into the second large intron of the Ftz-F1 (CG4059) transcription unit in antisense orientation very close to each other. All EST sequences overlap with the sequence of the predicted gene CG4059 (Ftz-F1) therefore EP(3)0447 and EP(3)25823 are homozygous viably integrated in the transcription unit of Ftz-F1. The gene Ftz-F1 is also represented by the ESTs DGC LD34889, Clot 3727_{—}2 and _{—}1 but their Gal4 promoters should direct ectopic expression of endogenous genes in the opposite direction in respect to the direction of CG4059 expression. Therefore, expression of the CG4059 could be effected by homozygous viable integration of EP(3)0447 and EP(3)25823 leading to increase of the energy storage triglycerides.

Ftz-F1 encodes for a gene that is predicted by GadFly sequence analysis programs (CG4059). No functional data described the regulation of obesity and metabolic diseases are available in the prior art for the genes with Acc. No. M63711, M98397, and AB019246, referred to as Ftz-F1 in the present invention.

### EXAMPLE 3

### Identification of Human Trp1, MCT, or Ftz-F1 Homologous Genes and Proteins

The present invention is describing a polypeptide comprising the amino acid sequence of Trp1 (GadFly Accession Number CG4758; GenBank Accession Number Z38100 for the cDNA, SPTREMBL Accession Number Q24559 for the protein). As shown in FIG. 3A, gene product of Trp1 is 55% homologous to the human translocation protein 1 (TLOC1; GenBank Accession Number NM_{—}003262 for the cDNA, NP_{—}003253 for the protein). A comparison (Clustal X 1.8) between the Trp1 proteins of different species (Drosophila isoforms dmTrpalt1 (Acc. No. AAF52847), dmTrpalt2 (Acc. No. AAF52848), dmTrp1 (Acc. No. Q24559), mouse mmtrp1 (Acc. No. BAB29058), and human hstrp1 (Acc. No. NP_{—}003253)) was conducted and is shown in FIG. 3B.

The predicted nucleic acid and amino acid sequences were searched in the publicly available databases, such as NCBI nr proteins, nt nucleotide, NCBI predicted proteins genome, EnsEMBL predicted proteins, NCBI human ESTs, human Genome (chromosome arms and htgs). In search of sequence databases, it was found, for example, that the Drosophila MCT protein (GadFly Accession Number CG8051) has 40% homology with human monocarboxylate transporter 3 protein (Accession Number: NP_{—}037488).

In particular, MCT protein and monocarboxylate transporter 3 protein share about 40% homology, starting between amino acid 80 and 263 of MCT protein, and 41% homology, starting between amino acid 423 and 567 of MCT protein.

In addition, it was found, for example, that the Drosophila MCT protein (GadFly Accession Number CG8051) has 41% homology with human solute carrier family 16 (monocarboxylate acid transporters), member 5 (Accession Number: NP_{—}004686). In particular, MCT protein and monocarboxylate acid transporter, member 5, protein share about 41% homology, starting between amino acid 81 and 274 of MCT protein. In addition, homologies to a genomic sequence AC040977 human chromosome 17 clone RP11-58.9P10 map 17 were found. This genomic sequence has not been further described yet. The sequence shows two translated regions (exons) matching regions in the MCT protein of the invention. Translation of basepairs 161145 to 161318 of AC040977 shows 56% homologies to amino acids 80 top 137 of the MCT protein of the invention, and translation of basepairs 162880 to 163122 of AC040977 shows 46% homologies to amino acids 183 to 263 of the MCT protein of the invention.

In addition, it was found, for example, that the MCT protein of the invention has homologies to other monocarboxylate acid transporters found in Drosophila (e.g., Accession Number: CG3456). Since protein domains are highly conserved, a protein domain analysis was conducted for the protein of the invention. We found that the protein of the invention (MCT, GadFly Accession Number CG8051 has a sugar transporter domain and a vesicular monoamine transporter domain (see FIG. 7A).

It was found, for example, that the MCT protein of the invention has at least ten transmembrane domains (FIG. 7B) which anchors the protein in cell membranes. Thus, the MCT protein is a membrane spanning protein likely to be associated with a variety of distinct biological processes in both prokaryotes and eukaryotes, for example in transport processes such as active transport of small hydrophilic molecules across the cytoplasmic membrane.

The present invention is describing a polypeptide comprising the amino acid sequence of Ftz-F1. The Drosophila Ftz-F1 alpha (Acc. No. AAA28542) and Ftz-F1 beta (Acc. No. AAA28915) proteins are identical except a short aminoterminal sequence. A comparison (Clustal X 1.8) between the Ftz-F1 protein of different species (human, mouse, and Drosophila) was conducted and shown in FIG. 11 (NP_{—}032076 refers to the mouse homolog; BAA34092 refers to human FTZ-F1 beta; FTF1 _DROME refers to the Drosophila isoform of FTZ-F1 alpha; AAA2891 refers to Drosophila FTZ-F1beta).

Using Pfam-protein analysis tools, it was found, for example, that the Ftz-F1 protein of the invention has at least two characteristic protein motifs domains. These motifs and targeting sequences are found throughout the whole Ftz-F1 famliy. Ftz-F1 has a zink finger domain of typ zf-c4 (amino acids 448-523 in Ftz-F1 alpha) and a ligand binding domain (type hormone_rec, amino acids 778-938 in Ftz-F1 alpha). Based upon homology, Ftz-F1 protein of the invention and each homologous protein or peptide may share at least some activity.

### EXAMPLE 4

### Expression of Polypeptides in Mammalian Tissues

For analyzing the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 percent humidity and a light/dark cycle of preferrably 14/10 hours. The mice were fed a standard chow (for example, from ssniff Spezialitäten GmbH, order number ssniff M-Z V1126-000). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition; for example, dexamethason and 3-isobutyl-1-methylxanthin), up to 10 days of differentiation, suitable aliquots of cells were taken every two days. Alternatively, mammalian fibroblast 3T3-F442A cells (e.g., Green & Kehinde, Cell 7: 105-113, 1976) were obtained from the Harvard Medical School, Department of Cell Biology (Boston, Mass., USA). 3T3-F442A cells were maintained as fibroblasts and differentiated into adipocytes as described previously (Djian, P. et al., J. Cell. Physiol., 124:554-556, 1985). At various time points of the differentiation procedure, beginning with day 0 (day of confluence and hormone addition, for example, Insulin), up to 10 days of differentiation, suitable aliquots of cells were taken every two days. 3T3-F442A cells are differentiating in vitro already in the confluent stage after hormone (insulin) addition.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH-Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

For the analysis of the expression of MCT 3 and MCT 5, taqman analysis was performed using the following primer/probe pairs (see FIG. 8):

| | |
|---|---|
| Mouse MCT 3 forward primer | (Seq ID NO: 3) |
| 5′-GAC CGT GCT TTC GTG GTG TAC-3′; | |
| Mouse MCT 3 reverse primer | (Seq ID NO: 4) |
| 5′-AGA TGG CCG GCA CAA AGA-3′; | |
| Mouse MCT 3 Taqman probe | (Seq ID NO: 5) |
| TCA CCA AGT TCC TGA TGG CAC TCG G | (5/6-FAM) |
| (5/6-TAMRA) | |
| Mouse MCT 5 forward primer | (Seq ID NO: 6) |
| 5′-CAT CAA CGG GCT CAC CAA TC-3′; | |
| Mouse MCT 5 reverse primer | (Seq ID NO: 7) |
| 5′-AGG CAA TAG CCC AGG AGC A-3′; | |
| Mouse MCT 5 Taqman probe | (Seq ID NO: 8) |
| TGC ACG GTG TCA GCC GAC TTC C | (5/6-FAM) |
| (5/6-TAMRA) | |

Taqman analysis revealed that MCT 5 is the more interesting homologue of the fly gene. In comparison to MCT 3, which is rather ubiquitously expressed, MCT 5 is highly restricted to colon and small intestine with almost no expression in adipogenic tissues (FIG. 8A). However, expression of MCT 5 in brown adipose tissue of genetically obese ob/ob mice is 50 fold upregulated compared to wild-type tissue (FIG. 8B). In addition, MCT 5 is also highly upregulated in liver of fasted and ob/ob mice, a tissue there MCT 5 is measurable expressed in the wild-type situation (FIG. 8B). These responses of MCT 5 are recapitulated in liver and BAT of mice hold under a high fat diet. Again, a strong upregulation is observed (FIG. 8B).

With regard to changes in expression intensity during the differentiation of preadipocytes to adipocytes, an increase of MCT 3 expression can be observed in 3T3-F442A cells. On the other hand, MCT 5 displays a clear reduction in relative signal intensity during the in vitro differentiation program of 3T3-F442A cells as well as TA1 cells (FIG. 8C).

For the analysis of the expression of Ftz-F1-1 and Ftz-F1-2, taqman analysis was performed using the following primer/probe pairs (see FIG. 12):

| | |
|---|---|
| Mouse Ftz-F1-1 forward primer | (Seq ID NO: 9) |
| 5′-CCT CCT GAG TCT CGC ACA GG-3′; | |
| Mouse Ftz-F1-1 reverse primer | (Seq ID NO: 10) |
| 5′-AAC TCC CGC TGA TCG AAC TG-3′; | |
| Mouse Ftz-F1-1 Taqman probe | (Seq ID NO: 11) |
| CTG GTG GTG AGG CTC CGT TCC CT | (5/6-FAM) |
| (5/6-TAMRA) | |
| Mouse Ftz-F1-2 forward primer | (Seq ID NO: 12) |
| 5′-GCC AAA AGC GGC TCT GAC-3′; | |
| Mouse Ftz-F1-2 reverse primer | (Seq ID NO: 13) |
| 5′-ATA AAG GTC TGG TCG GCC ATT-3′; | |
| Mouse Ftz-F1-2 Taqman probe | (Seq ID NO: 14) |
| AGC GCC CTT CAG CCT CCT CTG C | (5/6-FAM) |
| (5/6-TAMRA) | |

Taqman analysis revealed that Ftz-F1-1 and Ftz-F1-2 show interesting responses in their expression pattern in metabolic active tissues of different mouse models. Both show a rather restricted expression in wildtype tissues (FIG. 12A). However, this expression is under metabolic control: In genetically obese (ob/ob) mice, expression is strongly increase in BAT (Ftz-F1-1) and WAT and kidney (Ftz-F1-2). In addition, expression of Ftz-F1-2 is strongly induced in kidney and midbrain of fasted mice (FIG. 12B). In addition, Ftz-F1-2 shows a prominent increase in its expression in muscle and BAT of mice under a high-fat diet (FIG. 12B).

During the in vitro differentiation of adipogenic cell lines, rather weak overall expression levels of these proteins are observed. Nethertheless, we could demonstrate a down-regulation of Ftz-F1-1 expression during the differentiation of TA1 cells. In contrast, expression of Ftz-F1-2 is up-regulation during the in vitro differentiation of 3T3-F442A cells from preadipocytes to (FIG. 12C). Using commercially available human total RNA, we could demonstrate a clear expression of Ftz-F1-1 in human adipose tissue (FIG. 12D).

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the translocation protein, monocarboxylate transporter, or nuclear hormone receptor gene family or a polypeptide encoded thereby or a fragment or a variant of said nucleic acid molecule or said polypeptide or an antibody, an aptamer or another receptor recognizing a nucleic acid molecule of the translocation protein, monocarboxylate transporter, or nuclear hormone receptor gene family or a polypeptide encoded thereby together with pharmaceutically acceptable carriers, diluents and/or adjuvants.

2. The composition of claim 1, wherein the nucleic acid molecule is a vertebrate or insect Trp1, MCT, or Ftz-F1 nucleic acid, particularly a human Trp1, MCT, or Ftz-F1 nucleic acid such as Trp1 (GenBank Accession No. NM 003262), MCT (e.g., GenBank Accession No. NP 037488, Genbank Accession No. NP 004686, or GenBank Accession No. AC040977), or Ftz-F1 (GenBank Accession No. NM 003822, derived from GenBank Accession No. AF146343; or GenBank Accession No. NM 004959, derived from GenBank Accession No. U76388), or a Drosophila nucleic acid such as GenBank Accession Number Z38100; SEQ ID NO:1, GenBank Accession Number M63711, or M98397.

3. The composition of claim 1, wherein said nucleic acid molecule
(a) hybridizes at 66° C. in a solution containing 0.2×SSC and 0.1% SDS to the complementary strand of a nucleic acid molecule encoding the amino acid sequence of SPTREMBL Acc. No. Q24559, SEQ ID NO:2, GenBank Acc. No. AAA28542, or GenBank Acc. No. AAA28915; or human homologous nucleic acid molecule selected from GenBank Accession Numbers NM_{—}003262 (Trp1); NP_{—}037488 (MCT3); NP_{—}004686 (MCT5); NP_{—}003813 (FTZ-F1) or NP_{—}004950.2 (FTZ-F1);
(b) it is degenerate with respect to the nucleic acid molecule of (a);
(c) encodes a polypeptide which is at least 85%, preferably at least 90%, more preferably at least 95%, more preferably at least 98% and up to 99.6% identical to the amino acid sequence of SPTREMBL Acc. No. Q24559, SEQ ID NO:2, GenBank Acc. No. AAA28542, or GenBank Acc. No. AAA28915 or a human homologous protein selected from GenBank Accession Numbers NM_{—}003262 (Trp1); NP_{—}037488 (MCT3); NP_{—}004686 (MCTS); NP 003813 (FTZ-F1) or NP 004950.2 (FTZ-F1);
(d) differs from the nucleic acid molecule of (a) to (c) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a pharmaceutical composition.

13. The composition of claim 1 for the manufacture of an agent for detecting and/or verifying, for the treatment, alleviation and/or prevention of diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others, in cells, cell masses, organs and/or subjects.

14. Use of a nucleic acid molecule of the translocation protein, monocarboxylate transporter, or nuclear hormone receptor gene family or a polypeptide encoded thereby or a fragment or a variant of said nucleic acid molecule or said polypeptide or an antibody, anaptamer or another receptor recognizing a nucleic acid molecule of the translocation protein, monocarboxylate transporter, or nuclear hormone receptor gene family or a polypeptide encoded thereby for controlling the function of a gene and/or a gene product which is influenced and/or modified by an translocation protein, monocarboxylate transporter, or nuclear hormone receptor homologous polypeptide.

15. Use of the nucleic acid molecule of the translocation protein, monocarboxylate transporter, or nuclear hormone receptor gene family or a polypeptide encoded thereby or a fragment or a variant of said nucleic acid molecule or said polypeptide or an antibody, an aptamer or another receptor recognizing a nucleic acid molecule of the translocation protein, monocarboxylate transporter, or nuclear hormone receptor gene family or a polypeptide encoded thereby for identifying substances capable of interacting with an Trp1, MCT, or Ftz-F1 homologous polypeptide.

16. A non-human transgenic animal exhibiting a modified expression of an Trp1, MCT, or Ftz-F1 homologous polypeptide.

17. The animal of claim 16, wherein the expression of the Trp1, MCT, or Ftz-F1 homologous polypeptide is increased and/or reduced.

18. A recombinant host cell exhibiting a modified expression of an Trp1, MCT, or Ftz-F1 homologous polypeptide.

19. The cell of claim 18 which is a human cell.

20. A method of identifying a polypeptide involved in the regulation of energy homeostasis and/or metabolism of triglycerides in a mammal comprising the steps of
(a) contacting a collection of (poly)peptides with an Trp1, MCT, or Ftz-F1 homologous polypeptide or a fragment thereof under conditions that allow binding of said (poly)peptides;
(b) removing (poly)peptides which do not bind and
(c) identifying (poly)peptides that bind to said Trp1, MCT, or Ftz-F1 homologous polypeptide.

21. A method of screening for an agent which modulates the interaction of an Trp1, MCT, or Ftz-F1 homologous polypeptide with a binding target/agent, comprising the steps of
(a) incubating a mixture comprising
(aa) an Trp1, MCT, or Ftz-F1 homologous polypeptide, or a fragment thereof or a fragment thereof;
(ab) a binding target/agent of said Trp1, MCT, or Ftz-F1 homologous polypeptide or fragment thereof; and (ac) a candidate agent
under conditions whereby said Trp1, MCT, or Ftz-F1 polypeptide or fragment thereof specifically binds to said binding target/agent at a reference affinity;
(b) detecting the binding affinity of said Trp1, MCT, or Ftz-F1 polypeptide or fragment thereof to said binding target to determine an (candidate) agent-biased affinity; and
(c) determining a difference between (candidate) agent-biased affinity and the reference affinity.

22. A method of producing a composition comprising the (poly)peptide identified by the method of claim 20 or the agent identified by the method of this with a pharmaceutically acceptable carrier, diluent and/or adjuvant.

23. The method of claim 22 wherein said composition is a pharmaceutical composition for preventing, alleviating or treating diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others.

24. Use of a polypeptide as identified by the method of claim 20 or of an agent as identified by the method of this invention for the preparation of a pharmaceutical composition for the treatment, alleviation and/or prevention of diseases and disorders related to body-weight regulation, for example, but not limited to, metabolic diseases such as obesity, as well as related disorders such as adipositas, eating disorders, wasting syndromes (cachexia), pancreatic dysfunctions (such as diabetes mellitus), hypertension, arteriosclerosis, coronary artery disease (CAD), hypercholesterolemia, dyslipidemia, osteoarthritis, gallstones, cancer, e.g. cancers of the reproductive organs, sleep apnea, and others.

25. Use of a nucleic acid molecule of the translocation protein, monocarboxylate transporter, or nuclear hormone receptor family or of a fragment thereof for the preparation of a non-human animal which over-or underexpresses the Trp1, MCT, or Ftz-F1 gene product.

26. Kit comprising at least one of
(a) an Trp1, MCT, or Ftz-F1 nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and (g) an anti-sense oligonucleotide of the nucleic acid of (a).
